(19) United States
(12) Patent Application Publication
(10) Pub. No.: US 2025/0275763 A1
(43) Pub. Date: Sep. 4, 2025

(54) SYSTEMS AND DEVICES FOR TISSUE REPAIR

(71) Applicant: EDWARDS LIFESCIENCES CORPORATION, Irvine, CA (US)

(72) Inventors: Mauricio Guerrero, Hawthorne, CA (US); Meena Francis, Trabuco Canyon, CA (US); Brian Patrick Murphy, Irvine, CA (US); Murrad Mirza Kazalbash, Tustin, CA (US)

(21) Appl. No.: 19/213,147

(22) Filed: May 20, 2025

Related U.S. Application Data
(63) Continuation of application No. PCT/US2023/080294, filed on Nov. 17, 2023.
(60) Provisional application No. 63/427,082, filed on Nov. 21, 2022.

(51) Int. Cl. A61B 17/04; A61B 17/00; A61F 2/24

(52) U.S. Cl. CPC A61B 17/0469; A61B 17/0482; A61B 17/0485; A61B 17/0487; A61B 17/0491; A61B 2017/00367; A61B 2017/0472; A61F 2/2457; A61F 2/2466

(57) ABSTRACT

Systems and devices for treating prolapse/bulging tissue and/or for closing tissue are provided. The systems and devices for treating prolapse/bulging tissue can include the use of a vacuum catheter and set of needles of pins. The vacuum can be used to grasp the tissue and the set of needles and pins can be used constrain the tissue via suturing. The systems and devices for closing tissue can include suturing system comprising a plurality of needles. The needles can be used to traverse an opening in tissue, leaving being a set sutures that can be cinched to close the opening.

### CROSS-REFERENCES TO RELATED APPLICATIONS

This application is a continuation of International Patent Application No. PCT/US2023/080294, filed Nov. 17, 2023, which claims the benefit of U.S. Patent Application No. 63/427,082, filed Nov. 21, 2022, the entire disclosures all of which are incorporated by reference for all purposes.

### BACKGROUND

The native heart valves (i.e., the aortic, pulmonary, tricuspid, and mitral valves) serve critical functions in assuring the forward flow of an adequate supply of blood through the cardiovascular system. These heart valves can be rendered less effective by congenital malformations, inflammatory processes, infectious conditions or disease. Such damage to the valves can result in serious cardiovascular compromise or death. Treatment for such disorders can be done with the surgical repair or replacement of the valve during open heart surgery or with transvascular techniques for introducing and implanting prosthetic devices in a manner that is much less invasive than open heart surgery.

A healthy heart has a generally conical shape that tapers to a lower apex. The heart has four chambers: the left atrium, right atrium, left ventricle, and right ventricle. The left and right sides of the heart are separated by a wall generally referred to as the septum. The native tricuspid valve of the human heart connects the right atrium to the right ventricle native and the mitral valve of the human heart connects the left atrium to the left ventricle. The tricuspid and mitral valve each include an annulus portion, which is an annular portion of the native valve tissue surrounding the valve orifice, and a set of two or three leaflets (also referred to as cusps) that extend downward from the annulus into the ventricle.

When operating properly, the leaflets of the tricuspid valve or of the mitral valve function together as a one-way valve to allow blood to flow only from the atrium to the ventricle. The right atrium receives deoxygenated blood from the superior vena cava and inferior vena cava, whereas the left atrium receives oxygenated blood from the pulmonary veins. When the muscles of the atrium contract and the ventricle dilates, the blood that is collected in the atrium flows into the ventricle. When the muscles of the atrium relax and the muscles of the ventricle contract, the increased blood pressure in the ventricle urges the two leaflets together, thereby closing the one-way valve so that blood cannot flow back to the atrium and is instead expelled out of the ventricle. To prevent leaflets from prolapsing or flailing under pressure and folding back through the valve annulus toward the atrium, a plurality of fibrous cords called chordae tendineae tether the leaflets to papillary muscles.

One complication that can occur with heart valves is leaflet prolapse in which a portion the leaflet has extra tissue or stretches, resulting in a billowing of the tissue. Prolapse can have various causes and is implicated in several disorders, including Barlow's disease, Carpentier's type II mitral valve regurgitation, and heart arrythmias. Prolapse is more commonly associated with the mitral and tricuspid valves, but can occur on the aortic and pulmonary valve as well. Standard treatments for leaflet prolapse include surgical intervention and can involve chordal transfer, chordal shortening, artificial chordae prosthetics, and anterior leaflet resection. In some implementations, treatments can involve heart valve replacement or specific treatments to the particular pathology. For example, mitral valve regurgitation can be treated by clipping the anterior and posterior leaflet together at the site of coaptation.

Another complication that can occur in the heart is myocardial rupture, septal rupture, spontaneous coronary artery dissection (SCAD), aortic aneurysm, ventricular septal rupture, or other tear in cardiac tissue. Ruptures of heart muscle and the heart septum can arise from various complications such as byproduct of myocardial infarction or blunt force trauma. When associated with myocardial infarction, the rupture can occur during the heart attack or sometime afterward (typically within 5 to 10 days and up to one month). SCAD is an emergency condition that arises when a tear spontaneously occurs in an artery. If the tear does not heal on its own, an intervention may be needed. An aortic aneurysm can arise due to genetic features, tobacco smoking, high blood pressure, and/or atherosclerosis.

### SUMMARY

This summary is meant to provide some examples and is not intended to be limiting of the scope of the invention in any way. For example, any feature included in an example of this summary is not required by the claims, unless the claims explicitly recite the feature. Also, the features, components, steps, concepts, etc. described in examples in this summary and elsewhere in this disclosure can be combined in a variety of ways. Various features and steps as described elsewhere in this disclosure can be included in the examples summarized here.

In some implementations, a system (e.g., for closing tissue, for closing an opening in tissue, for treating prolapse, for treating bulging tissue, for treating an aneurysm, etc.) includes a transcatheter system that includes a vacuum catheter, a needle assembly, and a suture.

In some implementations, the vacuum catheter includes an inner lumen for providing a vacuum force and set of channels in a sidewall of the vacuum catheter.

In some implementations, the vacuum catheter includes a set of channels in a sidewall of the vacuum catheter that channels allow distal advancement and proximal retrieval of the needle assembly.

In some implementations, the needle assembly includes a needle and a pin for suturing the suture.

In some implementations, the vacuum catheter includes a set of ports. Each channel is connected with at least one port that faces the inner lumen.

In some implementations, the set of channels includes an initial channel and a final channel.

In some implementations, the set of channels includes one or more intermediate channels.

In some implementations, each intermediate channel is in connection with an entry port and an exit port. The entry port and the exit port are connected via a connector channel.

In some implementations, each connector channel comprises a slit-shaped aperture facing the inner lumen.

In some implementations, each entry port of each intermediate channel is across an exit port on the opposite of the inner lumen.

In some implementations, the initial channel comprises an exit port

In some implementations, the exit port of the initial channel is across an entry port of an intermediate channel.

In some implementations, the final channel comprises an entry port.

In some implementations, the entry port of the final channel is across an exit port of an intermediate channel.

In some implementations, each channel has a companion channel across the inner lumen to yield a set of pairs of channels.

In some implementations, each pair of channels of the set comprises a first channel that comprises an exit port and a second channel that comprises an entry port.

In some implementations, the exit port of the first channel is across the inner lumen from the entry port of the second channel.

In some implementations, the pin comprises a means for grasping the suture at a distal end of the pin.

In some implementations, the pin is composed of a material with shape memory.

In some implementations, the needle comprises an inner lumen capable of allowing the pin to traverse therethrough.

In some implementations, the needle comprises a distal end that is sharpened, heated, or provides radiofrequency ablation.

In some implementations, the transcatheter system is sterilized and stored within a container.

In some implementations, a method (e.g., for closing tissue, for closing an opening in tissue, for treating prolapse, for treating bulging tissue, for treating an aneurysm, etc.) includes advancing a transcatheter system to tissue to be treated. In some implementations, the transcatheter system includes a vacuum catheter, a needle assembly, and a suture.

In some implementations, the needle assembly includes a needle and a pin.

In some implementations, the vacuum catheter includes an inner lumen for providing a vacuum force and set of channels in a sidewall of the vacuum catheter. In some implementations, the set of channels allow distal advancement and proximal retrieval of the needle assembly.

In some implementations, each channel of the set comprises a port facing the inner lumen.

In some implementations, the tissue is contacted with the vacuum catheter.

In some implementations, a vacuum force is applied to the tissue via the inner lumen of the vacuum catheter such that the tissue is constrained therein.

In some implementations, the tissue is sutured with suture utilizing the needle assembly and the set of channels.

In some implementations, the tissue is sutured by distally advancing the first needle assembly through a channel of the set of channels and through its port to the tissue.

In some implementations, the tissue is sutured by traversing the tissue with the first needle assembly. The first needle assembly carries the suture such that it traverses the tissue.

In some implementations, the set of channels comprises an initial channel and a final channel.

In some implementations, the first needle assembly is distally advanced through the initial channel.

In some implementations, the first needle assembly is traversed through a port of the initial channel, and the tissue such that the first needle assembly carries the suture such that it traverses the port of the initial channel and the tissue.

In some implementations, the suture is grasped by a final pin within a final channel.

In some implementations, the final pin is proximally retracted such that the final pin carries the suture as it is proximally retracted.

In some implementations, a proximal end of the suture is secured to the tissue.

In some implementations, a distal end of the suture is secured to the tissue.

In some implementations, the proximal end of the suture is preassembled to include a knot or a pledget.

In some implementations, the final pin includes a means for tying off the distal end of the suture.

In some implementations, the set of channels comprises an intermediate channel. In some implementations, the intermediate channel is in connection with an entry port and an exit port. In some implementations, the entry port and the exit port are connected via a connector channel.

In some implementations, the first needle assembly is advanced through the entry port of the intermediate channel such that the first needle assembly carries the suture such that it traverses the entry port of the intermediate channel.

In some implementations, a second needle assembly is distally advanced through the intermediate channel. In some implementations, the second needle assembly includes a needle and a pin.

In some implementations, the suture is grasped with the pin of the second needle assembly within the intermediate channel.

In some implementations, the second needle assembly is traversed through the exit port of the intermediate channel and the tissue such that the second needle assembly carries the suture such that it traverses the exit port of the intermediate channel and the tissue.

In some implementations, the cardiovascular system is reconstituted with blood or saline.

The above method(s) can be performed on a living animal or on a non-living simulation, such as on a cadaver, cadaver heart, anthropomorphic ghost, simulator (e.g., with the body parts, tissue, etc. being simulated), etc.

In some implementations, a system for transcatheter suturing includes a suturing catheter. In some implementations, the suturing catheter includes an elongated aperture at the distal end of the suturing catheter and a closed distal tip.

In some implementations, the system also includes a suturing system situated within the elongated aperture.

In some implementations, the suturing system includes a plurality of needles. Each having a needle tip and an inner lumen with a traversing suture within the inner lumen. Each of the needles are attached to a needle holder.

In some implementations, the needle holder is an elongated rod.

In some implementations, the suturing catheter includes a flex hinge positioned at the proximal end of the elongated aperture.

In some implementations, the system includes a suturing driver situated proximally positioned relative to the suturing the system.

In some implementations, the suturing driver comprises a torqueing rod that is in connection with the needles via the needle holder.

In some implementations, circular turning of the torqueing rod results in the needles turning in a circular motion.

In some implementations, the suturing driver is within the suturing catheter.

In some implementations, the suturing driver is extracorporeal.

In some implementations, each traversing suture includes a loop at the ends of the suture.

In some implementations, the suturing catheter includes a final suture channel and a plurality of notches within its side wall.

In some implementations, each notch is designed to fit and position the suture loop therein such that the suture loop is aligned with an axis of the final suture channel.

In some implementations, the system includes a final suture configured traverse the final suture channel and each suture loop when aligned with the axis of the final suture channel.

In some implementations, the system includes a pledget for cinching and securing the final suture.

In some implementations, the system includes a suture positioning shaft that is situated near and extended along the needle holder.

In some implementations, the suture positioning shaft is capable of positioning a suture loop within its respective notch via a circular motion of the suture positioning shaft.

In some implementations, each needle includes a suture positioning channel. The positioning shaft traverses through each suture positioning channel.

In some implementations, each suture positioning channel limits the circular motion of the suture positioning shaft.

In some implementations, the suture positioning shaft traverses through the suture loops that are to be positioned within its respective notch.

In some implementations, each needle of the plurality of needles comprises a tip that is sharpened, heated, or provides radiofrequency ablation.

In some implementations, the system includes an extracorporeal controller in connection with the suturing system.

In some implementations, the suturing system is sterilized and stored within a container.

In some implementations, a method is for suturing tissue. In some implementations, a transcatheter system is advanced to a site to be sutured. In some implementations, the transcatheter system includes a suturing catheter, a suturing system, and/or a suturing driver.

In some implementations, the suturing catheter includes an elongated aperture at the distal end of the suturing catheter, a closed distal tip, and a final suture channel.

In some implementations, the suturing system is situated within the elongated aperture and includes a plurality needles. In some implementations, each needle has a needle tip and an inner lumen with a traversing suture within the inner lumen. In some implementations, each of the needles are attached to a needle holder.

In some implementations, each traversing suture has first suture loop on one end of the suture and second suture loop on the other end of the suture.

In some implementations, the suturing driver is situated proximally positioned relative to the suturing the system.

In some implementations, the suturing driver comprises a torqueing rod that is in connection with the needles via the needle holder.

In some implementations, the elongated aperture and suturing system is situated over the site to be sutured.

In some implementations, the torqueing rod is turned in a first circular motion such that the plurality of needles penetrates and traverses through the tissue site to be sutured.

In some implementations, the circular motion of the needles results in the each of the traversing sutures to traverse through the tissue site to be sutured.

In some implementations, each first suture loop is positioned to align with the final suture channel.

In some implementations, a final suture is passed through the final suturing channel and through each first suture loop.

In some implementations, the torqueing rod is turned in a second circular motion that is opposite to the first circular motion such that the plurality of needles retreat back through the tissue site to be sutured.

In some implementations, the final suture that is traversed through each first suture loop keeps the plurality of traversing sutures traversed through the tissue site to be sutured.

In some implementations, each second suture loop is positioned to align with the final suture channel.

In some implementations, the final suture is passed through the final suturing channel and through each second suture loop.

In some implementations, the plurality of traversing sutures is cinched via the final suture.

In some implementations, the plurality of traversing sutures is cinched via a pledget.

In some implementations, the suturing system includes a suture positioning shaft that is situated near and extended along the needle holder. In some implementations, the suture positioning shaft traverses through the suture loops. In some implementations, the positioning of each first suture loop is positioned by moving the suture positioning shaft.

In some implementations, a transfemoral approach, a subclavian approach, a transapical approach, or a transaortic approach is utilized to reach the tissue site to be sutured.

In some implementations, the tissue site to be sutured is an injury.

In some implementations, the injury is a rupture, a tear, or an aneurism.

In some implementations, the tissue site to be sutured is an opening.

In some implementations, the opening is an opening to an appendage (e.g., an atrial appendage, a left atrial appendage, etc.) and the opening is closed according to the steps above.

In some implementations, the tissue site to be sutured is an incision.

In some implementations, the tissue site to be sutured is annular tissue of a native valve.

In some implementations, the native valve is regurgitant.

The above method(s) can be performed on a living animal or on a non-living simulation, such as on a cadaver, cadaver heart, anthropomorphic ghost, simulator (e.g., with the body parts, tissue, etc. being simulated), etc.

Any of the above systems, devices, apparatuses, components, etc. can be sterilized (e.g., with heat, radiation, ethylene oxide, hydrogen peroxide, etc.) to ensure they are safe for use with patients, and the above methods can comprise (or additional methods consist of) sterilization of one or more systems, devices, apparatuses, components, etc. herein (e.g., with heat, radiation, ethylene oxide, hydrogen peroxide, etc.).

### BRIEF DESCRIPTION OF THE DRAWINGS

The description and claims will be more fully understood with reference to the following figures and data graphs, which are presented as examples of the disclosure and should not be construed as a complete recitation of the scope of the disclosure.

FIGS. 1A and 1B provide views of a mitral valve.

FIGS. 2A and 2B provide views of an example leaflet prolapse of the mitral valve.

FIGS. 3A and 3B provide views of an example system for treatment of prolapse tissue.

FIG. 4 provides an overview method for treatment of prolapsed or bulging tissue via a catheter system.

FIGS. 5A1 to 5I provide an example detailed method for treatment of prolapsed or bulging tissue via an example catheter system.

FIGS. 6A and 6B provide example schematic depictions of suturing patterns that can be performed via an example catheter system.

FIGS. 7A and 7B provide views of a rupture within myocardial tissue as an example of a cardiovascular injury/opening.

FIGS. 8A to 8C provide views of an example system for repairing tissue via an example needle assembly delivered by catheter.

FIGS. 9A and 9B provide views of an example suturing driver usable in an example system for repairing tissue.

FIGS. 10A to 10D provide views of an example suturing system usable in an example system for repairing tissue.

FIGS. 11A1 to 11H provide an example detailed method for closing an opening in tissue via an example needle assembly delivered by catheter.

FIGS. 12A to 12D provide an example detailed method for performing annuloplasty via an example needle assembly delivered by catheter.

### DETAILED DESCRIPTION

The current disclosure details systems and devices for repairing tissue. The systems and devices can utilize a catheter design for minimal invasiveness and/or travel through the circulatory within the body. Accordingly, systems and devices can comprise a catheter that provides a means for translocating tools for repairing tissue.

Some of the systems and devices of the current disclosure are directed to repairing prolapsed or bulging tissue (e.g., leaflet prolapse, etc.), which can comprise a catheter-based vacuum and sets of needles and pins for suturing. These systems and devices can be utilized to reach the site of prolapsed or bulging tissue via a catheter delivery system and a vacuum can be applied to the prolapsed or bulging area to grasp and constrain the loose tissue. With the tissue constrained, a set of needles and pins and one or more sutures can be delivered to the site of prolapse or bulge. The needles and pins can stitch the constrained tissue such that it remains fixed in a constrained state. In some implementations, a portion of the constrained tissue can be resected or otherwise removed. In some implementations, the removed tissue is excess tissue that is not useful for leaflet function.

Some of the systems and devices of the current disclosure are directed to suturing via a minimally invasive technique, which can be utilized to repair/closure of a wound, injury, rupture, tear, and/or an incision of tissue, especially cardiac tissue, and/or to closing an opening (e.g., an opening in tissue, an opening in the side of a blood vessel, an incision in tissue, a rupture in tissue, a tear in tissue, an opening to an appendage, etc.), and/or to reduce suture together tissue to reduce size (e.g., reduction of a valve annulus). A catheter can be utilized to deliver suturing system to the site needing repair.

The suturing system can comprise a set of needles associated with a set of sutures. The sutures can include loops on one or both ends, which allow the suture to be gathered.

In some implementations, the set of needles can traverse injured tissue (e.g., a rupture, tear, incision, etc.) such that each suture has one end on both sides of the injury. In some implementations, the set of needles can traverse through any tissue (healthy or injured) for reduction of tissue surface area.

A final suture can be passed through the loops at the ends of each suture, gathering the sutures and tightening the sutures such that it closes the rupture or tear and/or reduces tissue surface area. The final suture can be tied off or otherwise secured, maintaining the gathered sutures in place.

The described systems, devices, and methods should not be construed as limiting in any way. Instead, the present disclosure is directed toward all novel and nonobvious features and aspects of the various disclosed systems and devices, alone and in various combinations and sub-combinations with one another. The disclosed systems, devices, and methods are not limited to any specific aspect, feature, or combination thereof, nor do the disclosed systems, devices, and methods require that any one or more specific advantages be present or problems be solved.

Various examples of tissue repair systems and components thereof are disclosed herein, and any combination of these examples can be made unless specifically excluded. For example, a vacuum system can be utilized with any needle and pin set, even if a specific combination is not explicitly described. Likewise, the different constructions and features of tissue repair systems can be mixed and matched, such as by combining the various components of the systems, even if not explicitly disclosed. In short, individual components of the disclosed systems can be combined unless mutually exclusive or physically impossible.

Although the operations of some of the disclosed methods are described in a particular, sequential order for convenient presentation, it should be understood that this manner of description encompasses rearrangement, unless a particular ordering is required by specific language set forth below. For example, operations described sequentially may in some cases be rearranged or performed concurrently. Moreover, for the sake of simplicity, the attached figures may not show the various ways in which the disclosed methods, systems, and apparatus can be used in conjunction with other systems, methods, and apparatus.

The terms “proximal” and “distal” as used throughout the description relate to a catheter system axis, in which the end where the procedure is performed is the distal end and the opposite end where the catheter system is controlled is the proximal end. Accordingly, the distal end of the catheter system is the leading end that first traverses into the body and first reaches the procedure site. Conversely, the proximal end of the catheter system is the portion that remains extracorporeal. Likewise, a distal movement along the catheter axis would be movement of a component in a direction towards a site of procedure and a proximal movement along the catheter axis would be movement of a component in an opposite direction. Although these terms have a relationship with a site of procedure, it is to be understood that these terms are used for reference and the site of procedure does not need to be present when interpreting the components or movements of the devices and systems described herein.

Various systems and devices for tissue repair are utilized for the purpose of performing a procedure within a recipient. Recipients can be a living animal or on a non-living simulation. Recipients include (but are not limited to) patients, animal models, cadavers, simulator (e.g., body parts, tissue, etc. being simulated), or anthropomorphic phantoms. Accordingly, in addition to methods of treating patients, the systems and devices can be utilized in training or other practice procedures upon animal models, cadavers, simulators, or anthropomorphic phantoms.

The described systems and devices can be sterilized, which can be performed using gamma irradiation, gas plasma, aldehydes, ethylene oxide, and/or e-beam. The systems or devices can be further treated with a formaldehyde bioburden reduction process. After preparation, the systems and devices can be stored and/or packaged within a container, which can be hermetically sealed or otherwise kept sterile.

For the purpose of understanding leaflet prolapse, the mitral valve is utilized as an example in the following figures and description. It is to be understood that leaflet prolapse can occur in any heart valve and thus reference to the mitral valve and treatments thereof are provided as an example of any of the valves, including the tricuspid valve, pulmonary valve, or aortic valve. In other words, any of the methods, devices, or description herein related to treatment of the mitral valve can be utilized and/or adapted to be utilized on the tricuspid valve, pulmonary valve, or aortic valve.

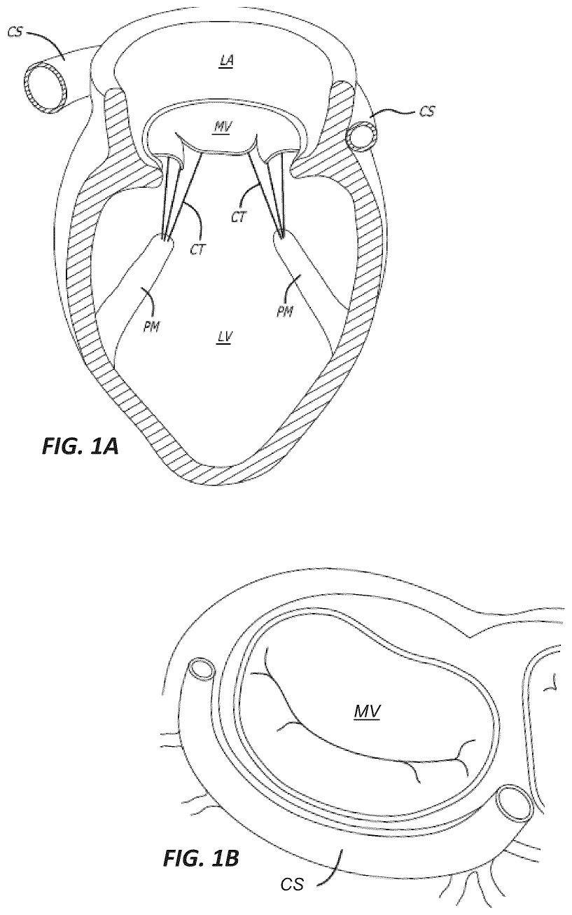

FIG. 1A is a coronal-plane view within the left chambers sectioning through the coaptation area of the mitral valve and FIG. 1B is a traverse-plane view within the left atrium superior to the mitral valve. The left ventricle (LV) is separated from the left atrium (LA) mitral valve (MV). Each of the four valves of the heart has flexible leaflets extending inward across the respective orifices that come together or “coapt” in the flowstream to form the one-way, fluid-occluding surfaces. Accordingly, referring back to the left chambers, oxygenated blood is brought to the left atrium from the pulmonary vein (not shown) and then transferred across the mitral valve into the left ventricle. The left ventricle pumps the oxygenated passing through the aortic valve, into the aorta, and throughout the body.

Also shown in FIG. 1A are the papillary muscles (PM), which are attached to the left ventricle wall and connected to the mitral valve (MV) leaflets via the chordae tendineae (CT). These muscles and cords assist in the function of the mitral valve (MV) to open the leaflets to form an aperture, to coapt the leaflets to close the aperture, and to maintain leaflet shape and position. The coronary sinus (CS), which is a vasculature that surrounds the left ventricle.

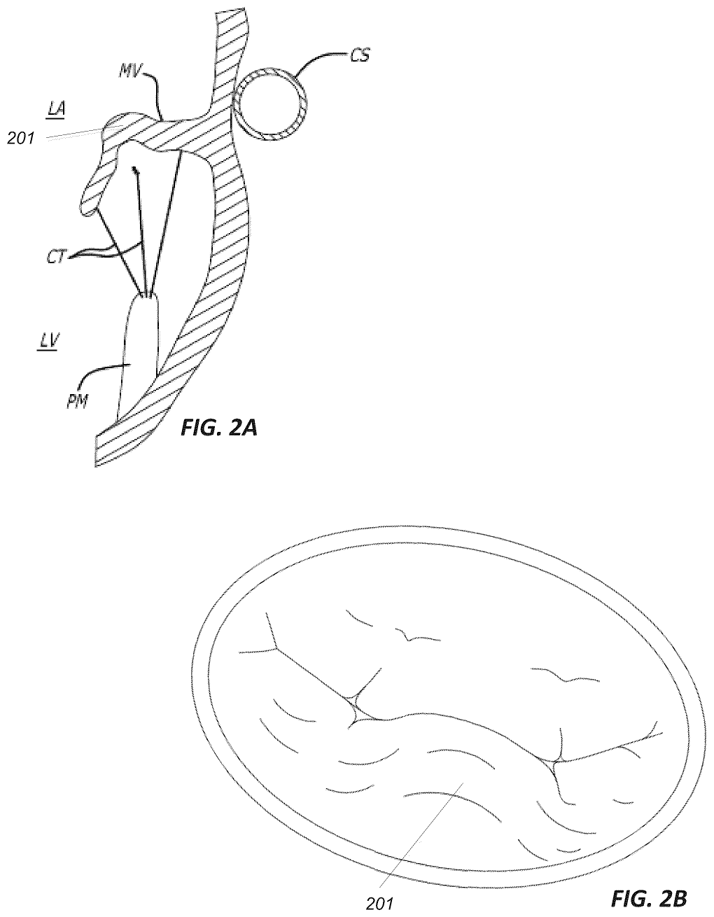

FIG. 2A and 2B provide an example of leaflet prolapse. Leaflet prolapse can occur when a portion of the leaflet protrudes backward. Prolapse can occur due to various conditions, including (but not limited to) papillary muscle (PM) and/or chordae tendineae (CT) dysfunction. In the example provided in FIG. 2A, breaks in the chordae tendineae (CT) result in leaflet prolapse. Leaflet prolapse can also occur due to the chordae tendineae (CT) stretching out.

Systems and devices (e.g., for treating leaflet prolapse, for treating bulging tissue, for treating aneurysm, for tissue repair, for valve repair, for tissue closure, etc.) comprise a catheter system such that it can be utilized within minimally invasive procedures and/or traverse through the circulatory system to reach the site of procedure. The systems and devices can comprise a vacuum to grasp and constrain tissue (e.g., prolapsed tissue of a leaflet, bulging tissue of an aneurysm, other tissue, etc.). The systems and devices can further comprise a set of needles, pins, and sutures to suture tissue such that it remains constrained.

The vacuum can be a catheter, which can be delivered to the site of procedure. In some implementations, a delivery catheter comprises an outer sheath and an inner sheath, wherein the inner sheath comprises the vacuum catheter. In some implementations, when the delivery catheter reaches the site of procedure, the vacuum of the inner sheath can be advanced beyond and out of the outer sheath to perform the procedure.

In some implementations, the vacuum catheter comprises a set of channels within the sidewall of the vacuum. The set of channels can provide a pathway for needles, pins and sutures to traverse to the procedure site. When the needles, pins and sutures traverse to the distal end of the vacuum catheter, ports within the vacuum sidewall provide a means for the needles and pins to penetrate and traverse the tissue (e.g., prolapsed tissue, etc.). The set of channels can comprise several channels, each having at least one port such that the set of needles and pins can traverse through the tissue several times to adequately suture the tissue (e.g., the prolapsed tissue) to ensure it is maintained constrained or in a constrained state.

The systems and devices (e.g., for treating prolapse, for treating bulging tissue, for treating an aneurysm, for closing tissue, for closing an opening in tissue, etc.) can be utilized on any leaflet of any of the four valves of the heart, including the mitral valve, the tricuspid valve, the pulmonary valve, and the aortic valve. Any transcatheter approach can be utilized to reach the tissue to be treated (e.g., leaflet prolapse, etc.), such as (for example) a transfemoral, subclavian, transapical, or transaortic approach.

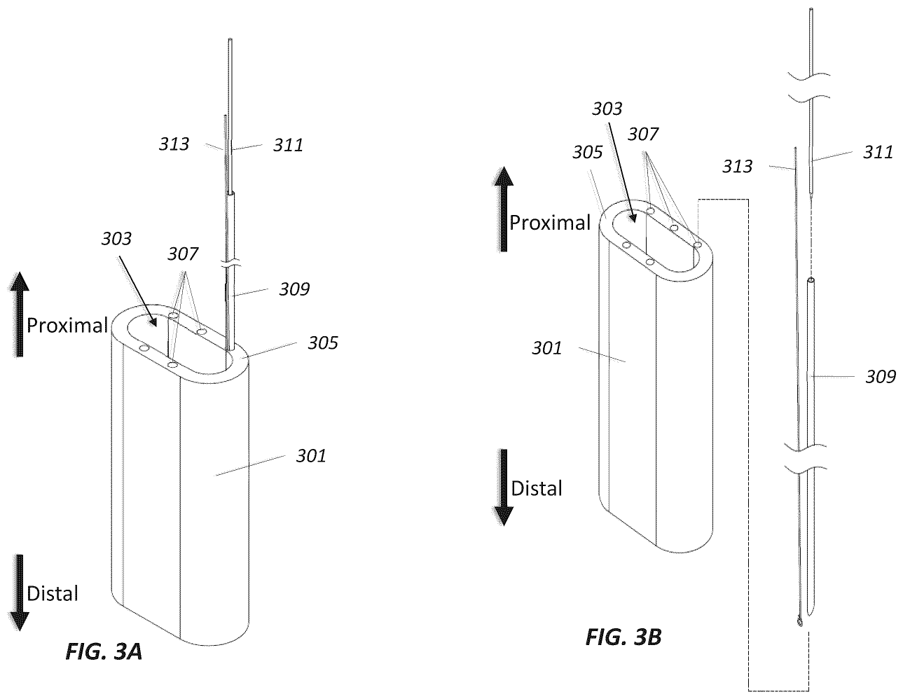

Provided in FIGS. 3A and 3B is an example of a catheter system (e.g., for treating prolapse, for treating bulging tissue, for treating an aneurysm, for closing tissue, for closing an opening in tissue, etc.), in which the distal end of the catheter is shown. The system comprises a catheter 301 (which can be a vacuum catheter). Although not shown, catheter 301 can extend proximally to an extracorporeal control and may be provided as part of an inner sheath encased with an outer sheath.

In some implementations, catheter 301 is a vacuum catheter and has an inner vacuum lumen 303 for providing a vacuum surrounded by the catheter sidewall 305.

In some implementations, the catheter sidewall 305 contains five channels 307 for providing a pathway for a needle 309, a pin 311, and a suture 313. Although five channels are shown, it should be understood that the catheter side wall can comprise at least 2 and up to any number of channels capable of fitting within the side wall, where the at least two catheters are on opposite sides across the vacuum lumen.

Needle 309 contains a sharp edge at the distal tip that can allow for the needle to traverse through tissue. A needle can optionally contain an inner lumen that can allow for a pin and/or suture to travel therethrough. As shown, needle 309 contains an inner lumen and pin 311 travels therethrough. It should be understood, however, that a pin and/or suture can travel within a channel alongside a needle. At the distal end of pin 311 is means for grasping suture 313, such as a hook, loop, or clasping member. The needle and pin can be composed of a memory material such as nitinol such that it can bend and flex to traverse through the channels and tissue.

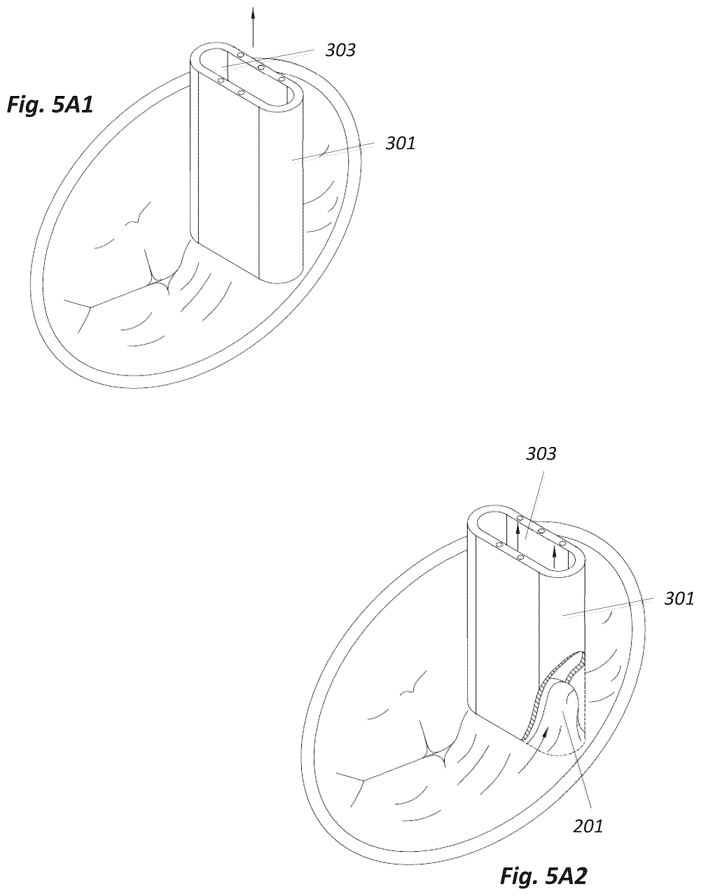

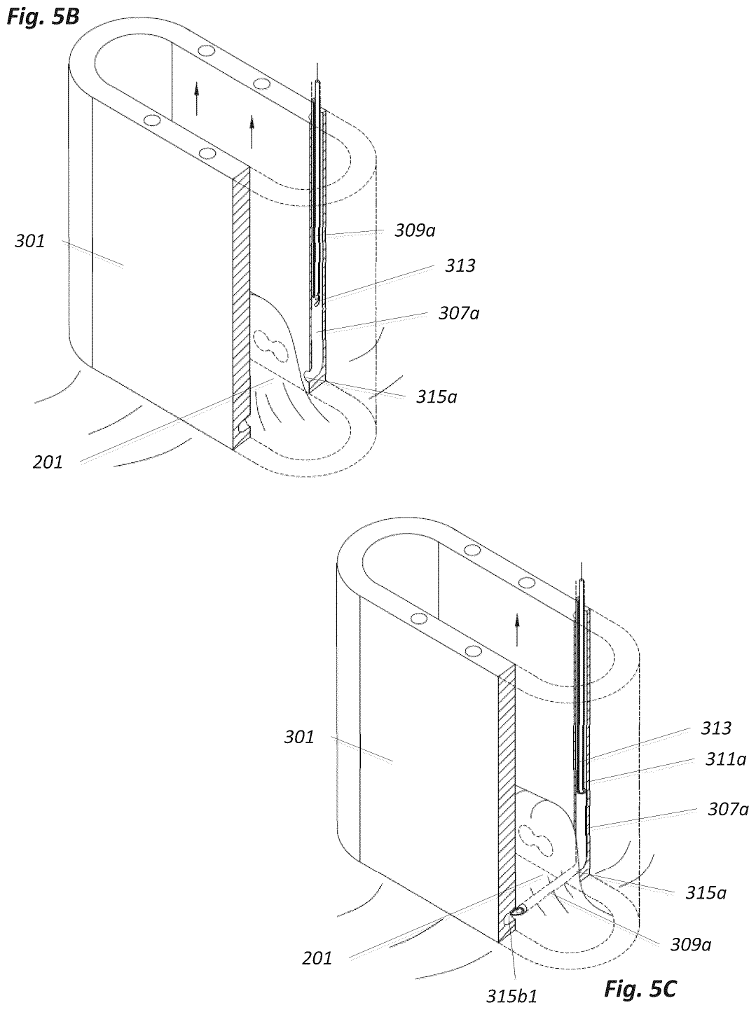

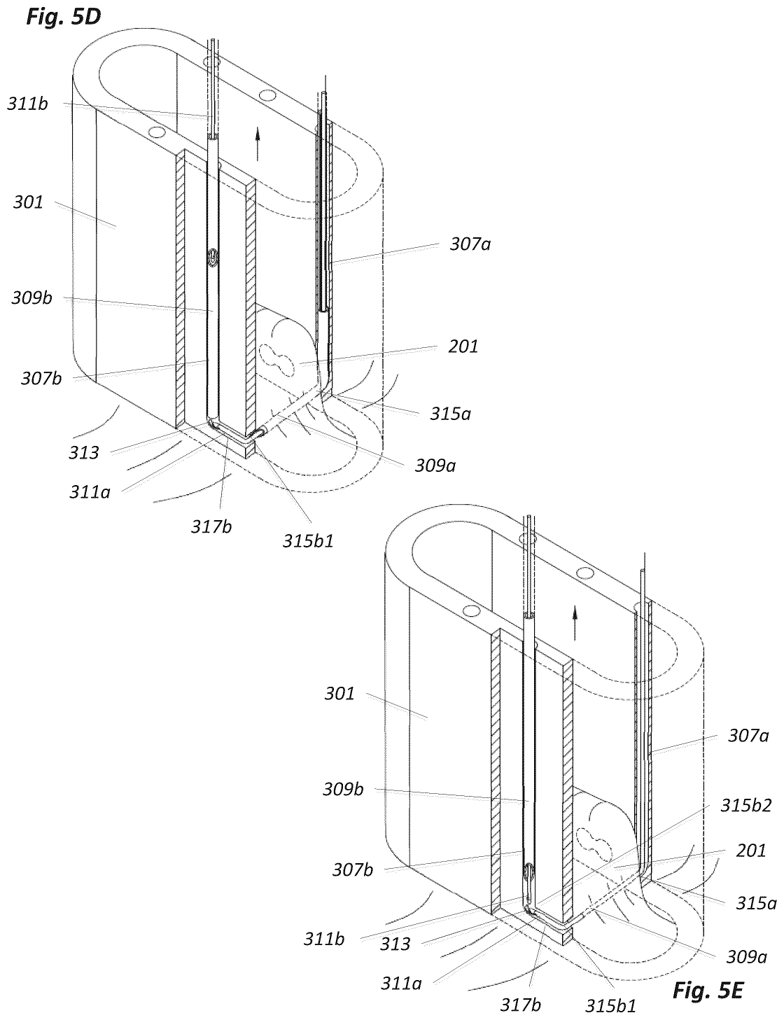

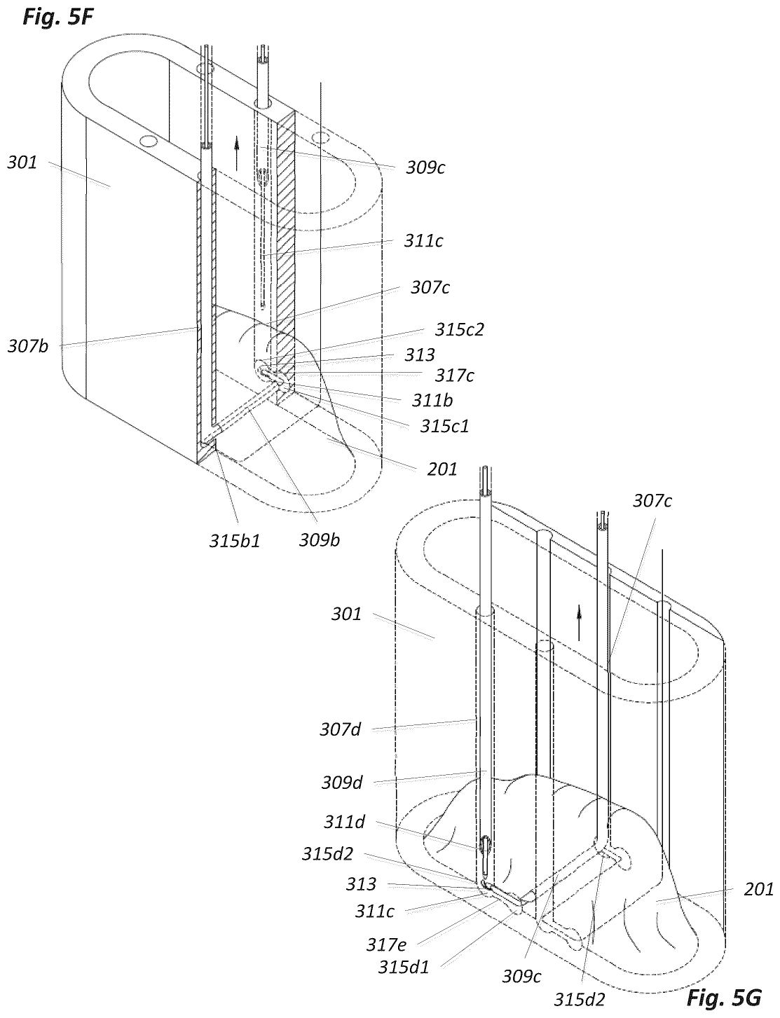

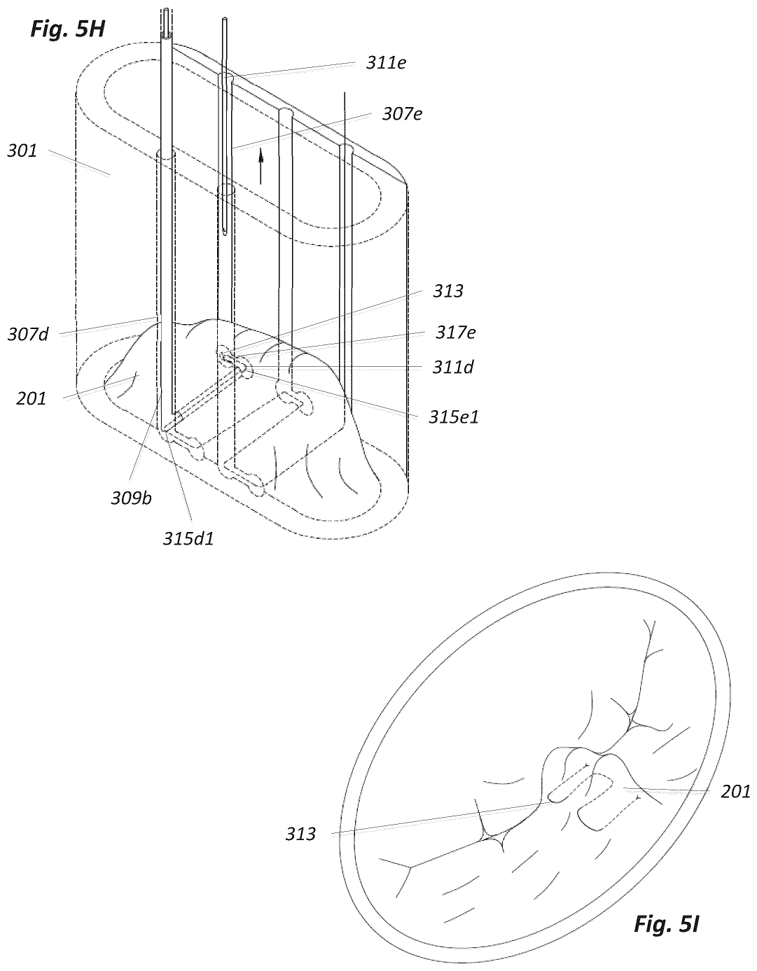

In some implementations, at the distal end of the vacuum catheter is a number of entry and exit ports 315 (see, e.g., FIG. 5H) that allow needle 309, pin 311, and suture 313 to penetrate and traverse the tissue (e.g., prolapsed tissue, etc.). Each channel 307 can have one or two ports. For instance, initial channel 307a can have a single port, for initiating penetration into the tissue. Intermediate channels 307b, 307c, and 307d can have two ports, one for receiving a traversed suture and a second for initiating penetration to traverse back across the tissue.

The two ports for each channel can be connected with a connector channel 317, which can include a slit-shaped aperture facing towards the lumen, which can allow a suture pass therethrough. Final channel 307e can also have one port for receiving a traversed suture. Although five channels are shown within the example of FIGS. 3A and 3B, a vacuum catheter can have at least two channels (e.g., an initial channel and a final channel), and can further optionally comprise any number of intermediate channels as permitted by the space availability within the side wall.

The vacuum catheter can further comprise a means for mitigating blood loss via the vacuum system. In some implementations, the vacuum catheter comprises a semipermeable membrane that allows vacuum suction but prevents blood and its constituents to flow through.

In some implementations, the vacuum system includes a recapture system in which blood and its constituents are captured and transferred back into the circulatory system. In some implementations, blood is allowed to be vacuumed into the vacuum system and the circulatory system is reconstituted with a blood transfusion (e.g., patient donates blood prior to procedure, which is utilized during the procedure for reconstitution).

Various implementations combine one or more means for mitigating blood loss. For example, in some implementations, a semipermeable membrane can be utilized to prevent blood constituents from being sucked into the vacuum but allows fluid from blood to be vacuumed; saline (or other biological fluid) is utilized to reconstitute the vacuumed fluid from the blood.

In some implementations, the vacuum catheter system extends proximally to a control system, as is appreciated in the art of transcatheter procedures. A clinician can utilize the control system to advance the transcatheter system through the body to the site where the procedure is to be performed. Further, the control system can advance and retract the vacuum in the distal and proximal direction in reference to an outer sheath. In some implementations, the control system can be utilized to control the vacuum power and strength of suction. In some implementations, the control system can further comprise a means for controlling the sets of needles and pins that travel through the channels within the vacuum sidewall. In some implementations, the control system can further comprise a means for securing or tying off the suture ends.

While a specific configuration of a system (e.g., for treating prolapse, for treating bulging tissue, for treating an aneurysm, for closing tissue, for closing an opening in tissue, etc.) is described above with reference to FIGS. 3A and 3B, it should be readily appreciated that various configurations of the system can be implemented in any of a variety of combinations of components. Accordingly, the specific configuration of the system described herein should be understood as not to be limited to any specific configuration, but instead can be implemented in any configuration capable of treating tissue (e.g., repairing leaflet prolapse, repairing bulging tissue, repairing aneurysm, closing an opening in tissue, etc.) via a vacuum catheter.

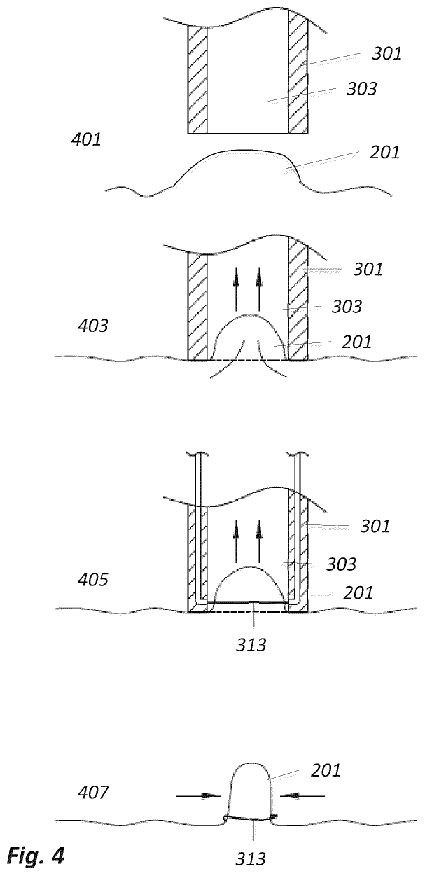

FIG. 4 provides an example method of how to use a catheter system to treat tissue (e.g., to repair a leaflet prolapse, to repair bulging tissue, to repair an aneurysm, to close an opening in tissue, etc.). The catheter system can be the system described in reference to FIGS. 3A and 3B or any alternate thereof, or another catheter system herein.

The various steps of this method can all be included and/or some may be omitted. And additional steps not described here are also possible. Steps can be done in different orders as well.

At step 401, the distal end of vacuum catheter 301 is advanced to the tissue 201 (e.g., to leaflet prolapsed tissue). At step 403, vacuum catheter 301 contacts tissue 201 and sucks/vacuums it into its lumen 303 to constrain the tissue.

At step 405, while tissue 201 is constrained by vacuum catheter 301, the set of needles, pins, and suture 313 are traversed through vacuum catheter sidewall and further through the tissue (e.g., through prolapsed tissue, etc.). At step 407, suture 313 is tied off or otherwise secured such that tissue 201 remains constrained or in a constrained state.

Vacuum catheter 301 is removed and rescinded back via the catheter delivery system.

In some implementations, when the tissue is bound, the excess tissue is resected or otherwise removed.

Provided in FIGS. 5A1 to 5I is an example method utilizing an example catheter system (e.g., a valve repair catheter system, a vacuum catheter system, etc.) to treat tissue. In the example illustrated, the system is used to repair a leaflet prolapse. The method utilizes the catheter system described in FIGS. 3A and 3B, but it should be understood that other catheter systems having similar design elements including a suturing system can be utilized. Further, while repair of leaflet prolapse is shown and described, other tissue treatments are possible with similar steps (e.g., treating other bulging tissue, treating aneurysm, closing tissue, suturing tissue, etc.)

FIGS. 5A1 and 5A2 are the same step with two different views where FIG. 5A2 provides a cut-away view allowing visualization of leaflet prolapsed tissue 201 within lumen 303 of catheter 301 (e.g., a vacuum catheter, etc.). At this step, catheter 301 is brought into contact with leaflet prolapsed tissue 201 of a mitral valve. The contact allows catheter 301 to grasp and constrain the prolapse within lumen 303 (e.g., via suction of the prolapse tissue into the catheter).

Any transcatheter approach to reach the mitral valve (or any other heart valve) can be utilized, such as (for example) a transfemoral, subclavian, transapical, transjugular, or transaortic approach.

As shown in FIG. 5B, a first needle assembly that includes needle 309a, pin 311a, and suture 313 is traversed down through initial channel 307a.

In some implementations, within the inner lumen of needle 309a is a pin 311a and along with pin 311a is a suture 313 that is held by a hook at the distal end of the pin. In some implementations, needle 309a, pin 311a, and suture 313 are traversed through initial channel 307a.

In FIG. 5C, needle 309a and pin 311a bends within initial channel 307a such that it can exit out of the channel via port 315a which opens towards prolapsed tissue 201, carrying suture 313 along with it. Needle 309a has a distal end that is capable to penetrate and traverse prolapsed tissue 201, and pin 311a and suture 313 can traverse the prolapse tissue along with the needle. The distal end can be sharpened and/or heated and/or provide radiofrequency ablation to assist penetration and traversing of tissue. In some implementations, after traversing prolapsed tissue 201, needle 309a, pin 311a and suture 313 enters into port 315b1.

FIG. 5D shows that pin 311a can be advanced beyond the distal tip of needle 309a, carrying suture 313 along with it. Pin 311a bends again to enter within connector channel 315b to enter in a space in connection with channel 307b. A second needle assembly that includes needle 309b pin 311b is traversed distally through channel 307b in the space in connection with connector channel 315b, coming into proximity with pin 311a and suture 313. Within this space, the distal hook of pin 311a can release its grasp of suture 313 and the distal hook of pin 311b can advance out of needle 311b to grasp suture 313, transferring the distal end of the suture the second needle assembly (FIG. 5E).

In some implementations, upon transferring of suture 313 to pin 311b of the send needle assembly, needle 309a and pin 311a can be proximally retracted back through channel 307a. Further, pin 311b can be proximally retracted back into needle 309b.

In some implementations, with pin 311b grasping suture 313 and retracted back within needle 309b, the second needle assembly can exit out of port 315b2, penetrate through prolapsed tissue 201, and enter port 315c1.

In some implementations, the process of transferring suture 313 from the second needle assembly to a third needle assembly is performed in the same manner as the transfer between the first and second needle assemblies (FIG. 5F). Accordingly, pin 311b distally advanced out the distal end of needle 309b, bends and travels through connector channel 317, carrying suture 313 into a space in connection with channel 307c. Pin 311c of the third needle assembly is distally advanced out of needle 309c into the connection space to grasp suture 313.

In some implementations, the third needle assembly (needle 309c and 311c) can be the same as the first needle assembly (309a and 311a) reused within channel 307c. Alternatively, the third needle assembly is unique to the first and second needle assemblies.

In some implementations, upon pin 311c grasping suture 313, it can be proximally retracted into needle 309c. The third needle assembly can then exit port 315c2 and traverse through prolapsed tissue 201 and enter port 315d1. Here, the process of transferring suture 313 from the third needle assembly to a fourth needle assembly is performed in the same manner as the prior transfers between needle assemblies (FIG. 5G). Accordingly, pin 311d grasps suture 313, proximally retracts into needle 309d, and the fourth needle assembly distally advances to exit port 315d2, to traverse through prolapsed tissue 201, and to enter port 315e1.

In some implementations, the fourth needle assembly (needle 309c and 311c) can be the same as the first needle assembly (309a and 311a) or the second needle assembly (309b and 311b) reused within channel 307d. Alternatively, the fourth needle assembly is unique to the first, second, and third needle assemblies.

In some implementations, to finalize the procedure, pin 311d can distally advance out of needle 319, travel through connector channel 317e into a space that connects with channel 307e.

In some implementations, a final assembly comprising pin 311e can be distally advanced within channel 307e into the same space to capture the suture (FIG. 5H). The final assembly can comprise a means for tying off, securing a pledget to, or otherwise securing the distal end of suture 313. To secure the proximal end of suture 313, in some implementations, the proximal end is preassembled to include a knot, a pledget, or other means for securing the proximal end of the suture at the first incision point within prolapsed tissue.

In some implementations, the distal end of suture 313 can be pulled proximally within the final channel until taut prior to tying off or otherwise securing the distal end. In some implementations, a pin can be distally advanced within first channel 307a to tie off or otherwise secure the proximal end of suture 313. In some implementations, once the distal and proximal ends of suture 313 are secured, the vacuum can be removed from the prolapsed tissue, resulting in the prolapses tissue being constrained by suture 313 (FIG. 5I).

In some implementations, once the prolapsed tissue is constrained by the suture, the procedure is completed. In some implementations, the excess tissue of the constrained prolapsed tissue is excised, ablated, or otherwise removed. Standard catheter techniques and catheter surgical tools can be utilized to excise, ablate, or otherwise remove and capture the excess tissue.

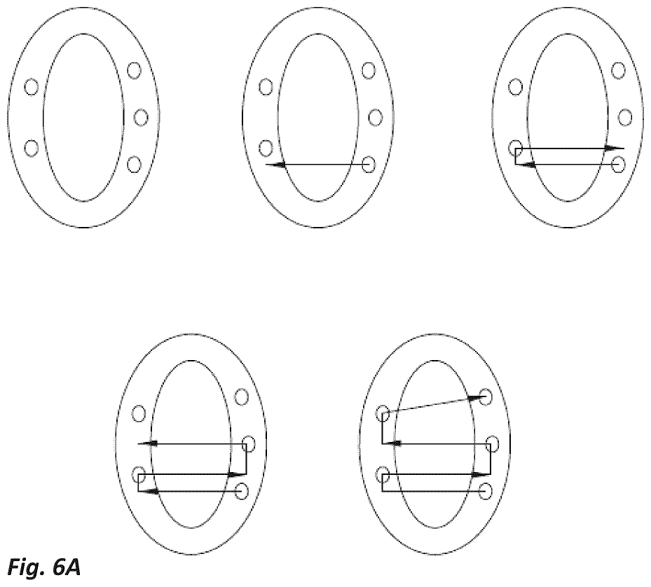

While the method described with reference to FIGS. 5A1 to 5I describe a particular suturing pattern, it is to be understood that any suturing pattern for constraining tissue can be utilized, as allowed by the channel design of the vacuum catheter. Further, any number of sutures can be utilized. Provided in FIG. 6A is an example of a suturing pattern that matches the vacuum catheter channels design and procedure described in reference to FIGS. 5A1 to 51. FIG. 6B, however, provides an example of a different pattern that utilizes a different vacuum channel design and four individual sutures. As shown at initial step 650, vacuum catheter 651 includes eight channels 653, where each channel has a complimentary channel direction across vacuum lumen 655. This channel design allows for 4 suture cross-stitches to be performed.

As can be viewed at step 652, a first suture 657a can be distally advanced through channel 653a1, then traverse through vacuum lumen 655 (and tissue therein, which is not shown for sake of visual clarity) into a port in connection with channel 653a2. A pin can be distally advanced through channel 653a2 to the distal end of suture 657a to tie off or otherwise secure the first suture therein. Likewise, the proximal end of suture 657a can be tied off or otherwise secured at or near channel 653a1.

Steps 654, 656 and 658 are essentially repeated steps of 652 with a unique suture. Accordingly, at step 654 suture 657b is distally advanced through channel 653b1, advanced through a port in connection with channel 653b1, traversed though vacuum lumen 655, and advanced into a port in connection with channel 653b2. The distal and proximal ends of suture 657b can be tied off or otherwise secured. Likewise, at step 656 suture 657c is distally advanced through channel 653c1, advanced through a port in connection with channel 653c1, traversed though vacuum lumen 655, and advanced into a port in connection with channel 653c2. The distal and proximal ends of suture 657c can be tied off or otherwise secured. And finally, at step 658 suture 657d is distally advanced through channel 653d1, advanced through a port in connection with channel 653d1, traversed though vacuum lumen 655, and advanced into a port in connection with channel 653d2. The distal and proximal ends of suture 657d can be tied off or otherwise secured. The cross-stitching of four sutures results in containing tissue within the vacuum lumen.

Ruptures, tears, aneurisms, incisions, openings, etc. can occur or be in multiple locations within the cardiovascular system. Some common injuries include a rupture or tear in myocardial tissue, papillary muscle tear, a tear in the inner heart wall, a hole or tear in the septum, an aneurysm in the aorta, and an aneurysm within blood vessels. In some instances, there is a need to tighten tissue, especially tissue that has been stretched or bulged. For instance, an annulus of valve can stretch out the leaflets such that they are unable to completely coapt, resulting in valve regurgitation. Surgical intervention can be used to repair an injury, incision, close an opening and/or tighten tissue.

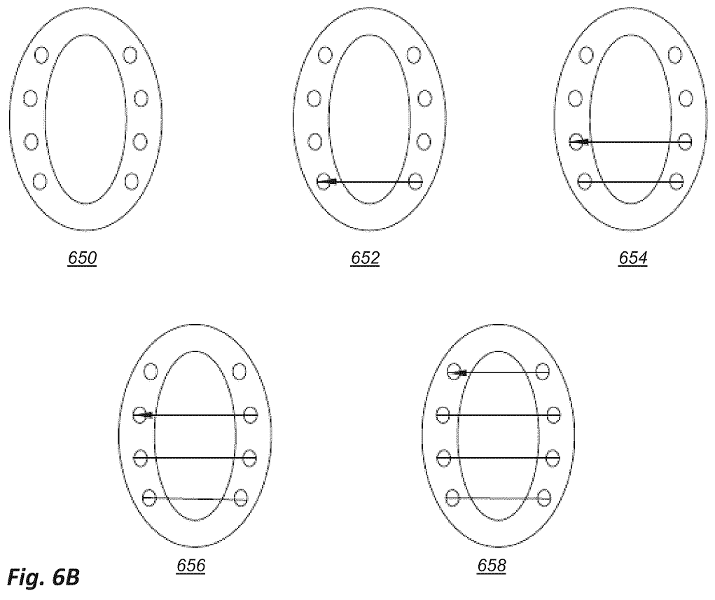

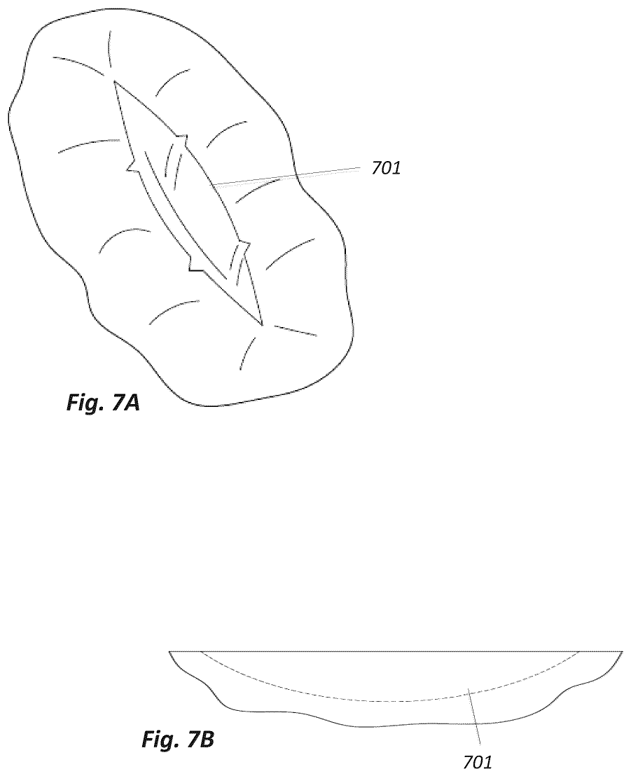

Illustrated in FIGS. 7A and 7B is an example of a cardiac tissue rupture 701, which is utilized as an example of an injury/opening that can be repaired by various systems and devices of the current disclosure. Similarly, incisions and/or other openings (e.g., an opening in tissue, an opening to an appendage, etc.) and/or loose tissue can be similarly repaired and/or closed and/or tightened. The systems, devices, etc. described herein can be utilized to treat a number of cardiovascular and other tissue issues such as rupture, tear, aneurysm, incision, a surgical flap, an opening, an injury, stretched tissue, etc. for which sutures can be used to repair the tissue.

In some implementations, the systems and devices (e.g., for repairing tissue injury, for closing an opening, for tightening tissue etc.) can comprise catheter suturing system. A transcatheter delivery system can deliver the catheter suturing system to a tissue site such that suturing system can suture and repair the tissue (e.g., closing of an opening, injury, tear, incision, etc.). In some implementations, the suturing system can comprise a needle assembly that comprises a plurality of needles and a suture for each needle.

In some implementations, the needle assembly can include a needle holder that can hold each of the needles to help the needles penetrate and traverse a tissue. In some implementations, the needle holder allows the needles to act in unison such that all the needles penetrate and traverse the tissue at the same time.

In some implementations, a driver system can be utilized to drive the needles to penetrate and traverse the tissue. In some implementations, the driver system utilizes a torqueing mechanism. Accordingly, the driver system can be turned in a circular direction to drive needles in a circular motion such that needles penetrate and traverse the tissue. As the needles traverse the tissue, each needle carries along a suture that is left within the tissue.

The systems and devices can further comprise a final suture that can be utilized to gather and tighten all the sutures traversing the tissue, resulting in a closing of and injury and/or opening and/or tightening of tissue.

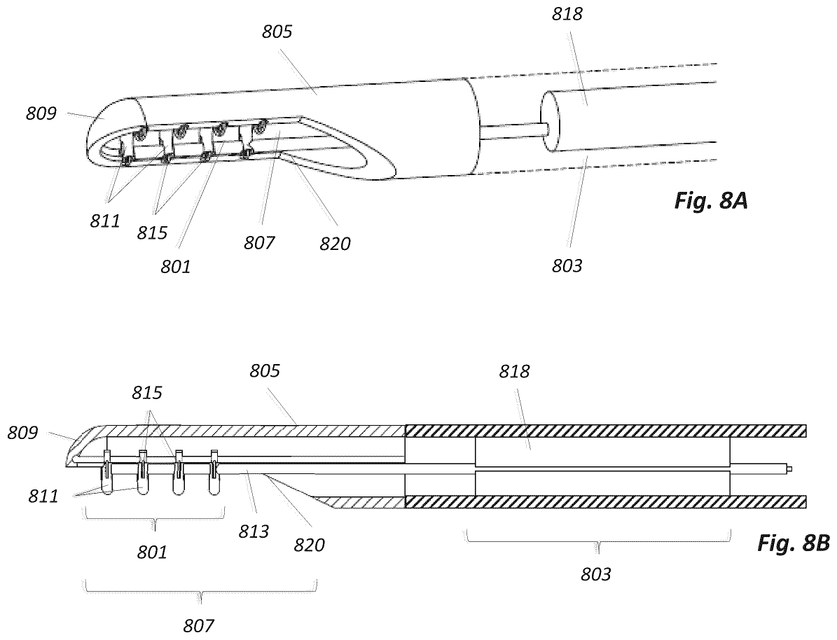

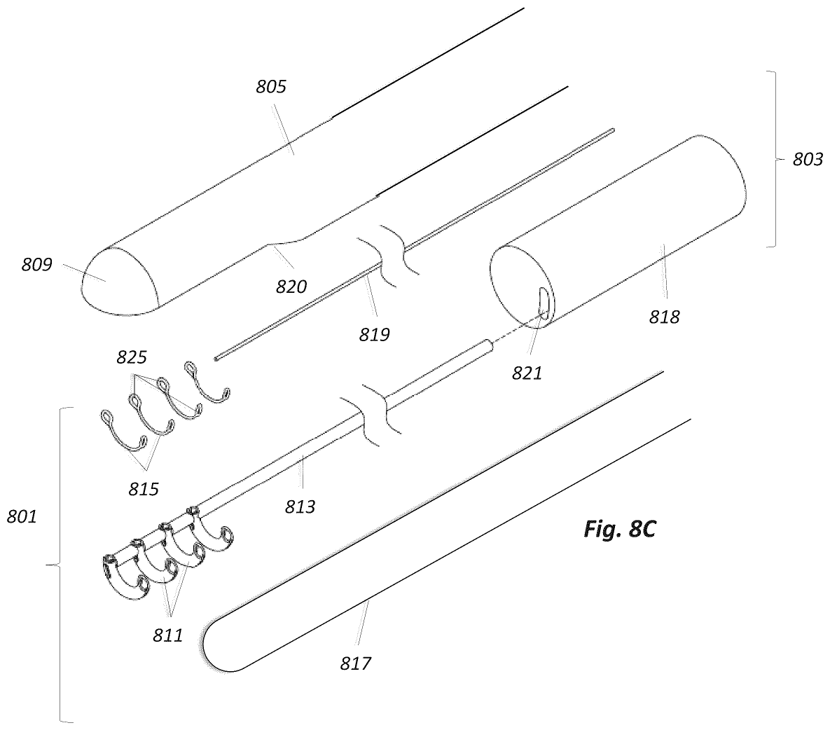

FIGS. 8A to 8C provide an example of system for suturing a wound, rupture, incision, opening, stretched tissue, etc. within the cardiovascular system and/or other tissue. In some implementations, the system comprises a suturing system 801 and a suturing driver 803 within a catheter 805.

In some implementations, the catheter 805 can comprise an elongated aperture 807 towards the distal end that spans along the distal-proximal axis. Elongated aperture 807 is large enough to allow exposure of suturing system 801. Catheter 805 can further comprise a rounded and closed distal tip 809. Proximal to the suturing system 801 is driver 803, which can be fully encapsulated within the catheter.

In some implementations, the suturing system can further comprise a plurality of needles 811 attached to a needle holder 813, a plurality of traversing sutures 815 for traversing tissue, an injury, a rupture, an incision, and/or an opening, and a final suture 817 for tightening traversing sutures 815 to close the injury, incision, and/or opening. Suturing driver 803 can comprise a torqueing rod 818 for driving needle holder 813 and needles 811 such that they penetrate and traverse the injured tissue. Suturing driver 803 can further comprise a suture positioning shaft 819 for position the plurality of traversing sutures.

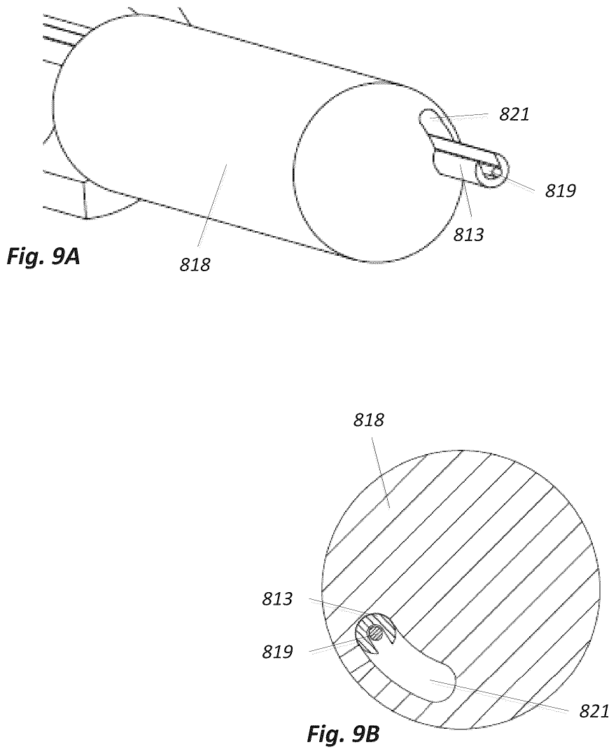

Provided in FIGS. 9A and 9B are detailed views of suturing driver 803. Suturing driver 803 comprises a rounded torqueing rod 818 that is capable of turning in a circular motion. The proximal end of needle holder 813 is attached or otherwise fixed to the outer edge of torqueing rod 818 such that when torqueing rod is turned in a circular motion, the needle holder turns in the circular motion as well. Needle holder 813 contains a groove along its axis that extends distally to suturing system 801. Groove allows suture positioning shaft 819 to situate therein, which also extends distally to suturing system 801. Within torqueing rod 818 is a channel 821 that allows for suture positioning shaft limited circular motion.

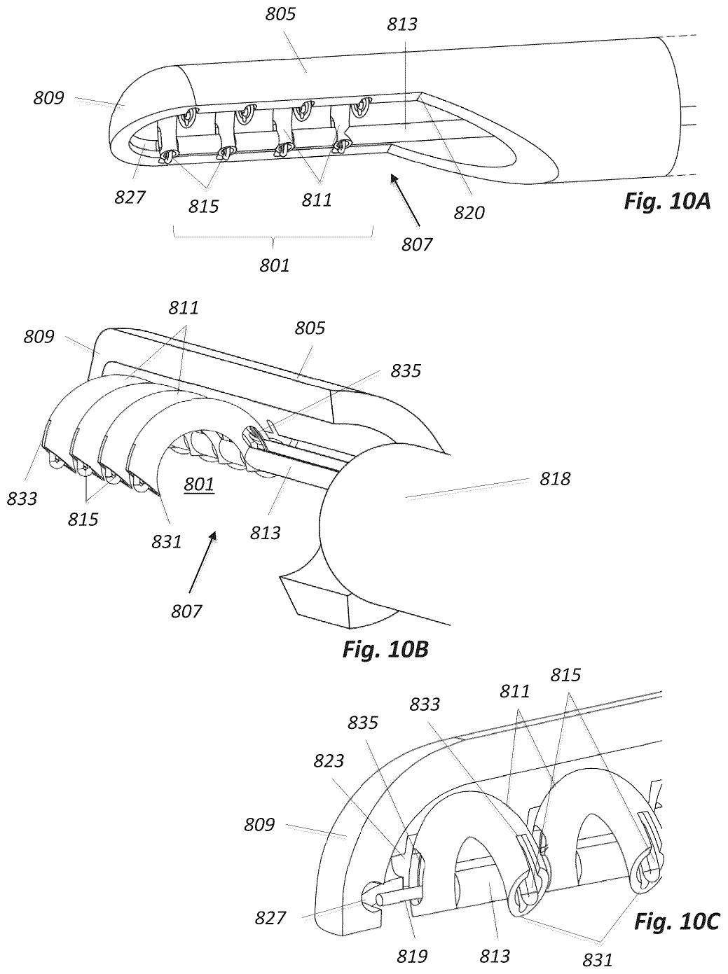

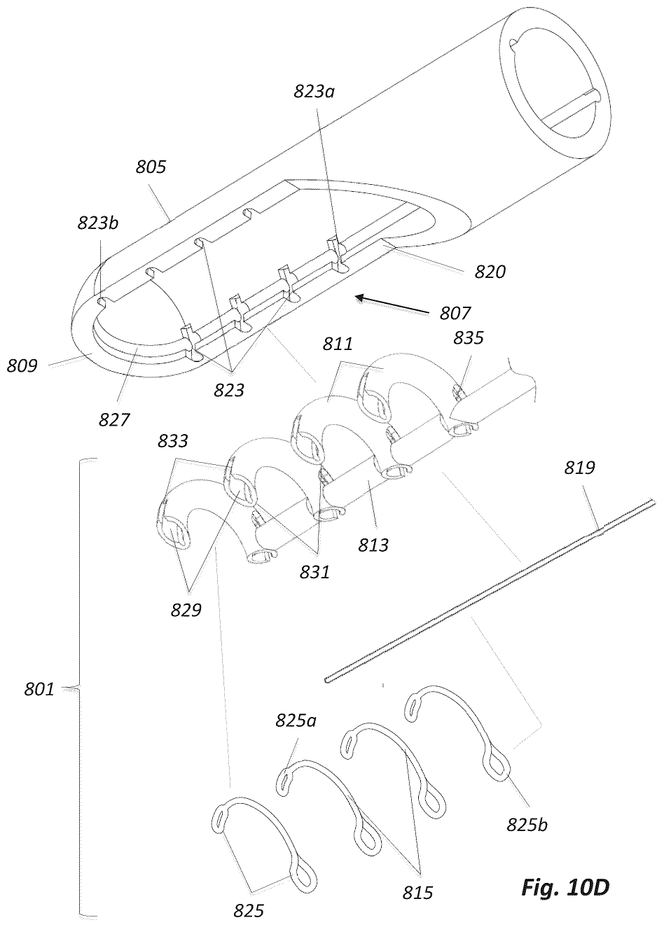

Provided in FIGS. 10A to 10D are detailed views of the distal end of catheter 805 and suturing system 801. Catheter 805 contains a flex hinge 820 that allows the catheter to flex and bend such that elongated aperture 807 and suturing system 801 can position and sit atop tissue, a wound, an injury, an incision, an opening, etc. to be closed and/or repaired. Within the catheter 805 are a number of grooves and notches that allow openings and pathways for sutures to fit within and/or travel therethrough. As can be viewed within elongated aperture 807 are suture loop notches 823 that allow suture loops 825 to fit therein.

In some implementations, a final suture channel 827 within the sidewall of catheter 805 and extends along the axis of the catheter. Final suture channel 827 further extends to and curves completely around and within closed distal tip 809. Suture loop notches 823 traverse final suture channel 827 such that when suture loops 825 are within the notches, final suture 817 can travel through the final suture channel and through each of the suture loops.

As will be discussed in more detail later, passing final suture 817 through each of suture loops 825 provides a means gathering and tightening traversing sutures 815 to tighten and/or close the tissue, injury, wound, rupture, incision, opening, etc.

In some implementations, suturing system has four needles 811 and each needle contains a traversing suture 815 within the needle's inner lumen 829. Each needle further has a tip 831 for penetrating and traversing though tissue, which can be beveled or otherwise have a means for penetrating and traversing tissue (e.g., sharp edge, heated, radiofrequency ablation, etc.). Across the opening of needle inner lumen 829 from needle tip 831 is a suture loop slit 833, that allows suture loops 825b open up and fit therein as part of the suturing mechanism, as described in greater detail below.

At the opposite side from needle tip 831, needles 811 are fixed to the distal portion of driving needle holder 813, which extends proximally to the torqueing rod 818. Where needles 811 are fixed to driving needle holder 813 is a suture positioning channel 829 within each needle. Suture positioning channel 835 allows for limited circular movement of suture positioning shaft 819 therein. Suture positioning shaft 819 traverses suture loops 825b on one side of the catheter. Suture positioning groove 835 allows movement of suture positioning shaft 819 to open up and to position suture loops 825b within suture loop notches 823, the mechanism to be described in more detail below.

In some implementations, the transcatheter-based system for suturing extends proximally to a control system, as is appreciated in the art of transcatheter procedures. A clinician can utilize the control system to advance the transcatheter system through the body to the site where the procedure is to be performed. Further, the control system can advance and retract the suturing system in the distal and proximal direction in reference to an outer sheath. In some implementations, the control system can be utilized to control the suturing system and suturing driver. In some implementations, the control system can further comprise a means for advancing and/or cinching the final suture. In some implementations, the control system can further comprise a means for securing the final suture.

While a specific configuration of a system for suturing is described above with reference to FIGS. 8A and 10D, it should be readily appreciated that various configurations of the system can be implemented in any of a variety of combinations of components. Accordingly, the specific configuration of the system described herein should be understood as not to be limited to any specific configuration, but instead can be implemented in any configuration capable of treating tissue via a suturing system.

Systems and devices for repairing and/or closing tissue can be delivered to the site of tissue via catheter. In some implementations, the catheter delivery system can be traversed through the circulatory system to an injury, incision, opening, stretched tissue, regurgitant valve, etc. occurring within cardiovascular system, including the heart and arteries. Any transcatheter approach can be utilized to reach the injury, incision, opening, etc., such as (for example) a transfemoral, subclavian, transapical, transjugular, or transaortic approach. In some implementations, the opening is an opening to an appendage (e.g., an atrial appendage, a left atrial appendage, etc.) and the opening is closed according to the steps below

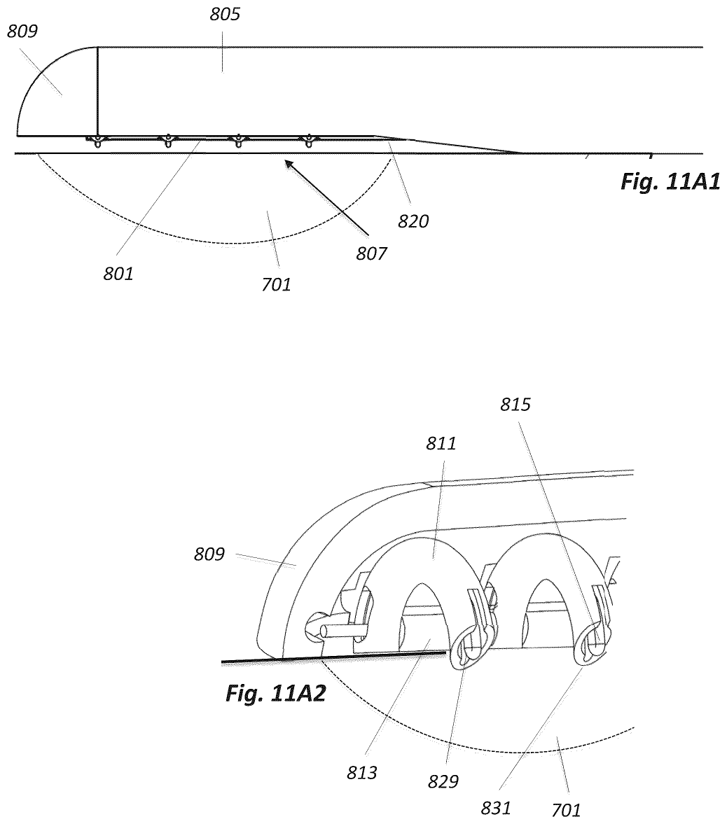

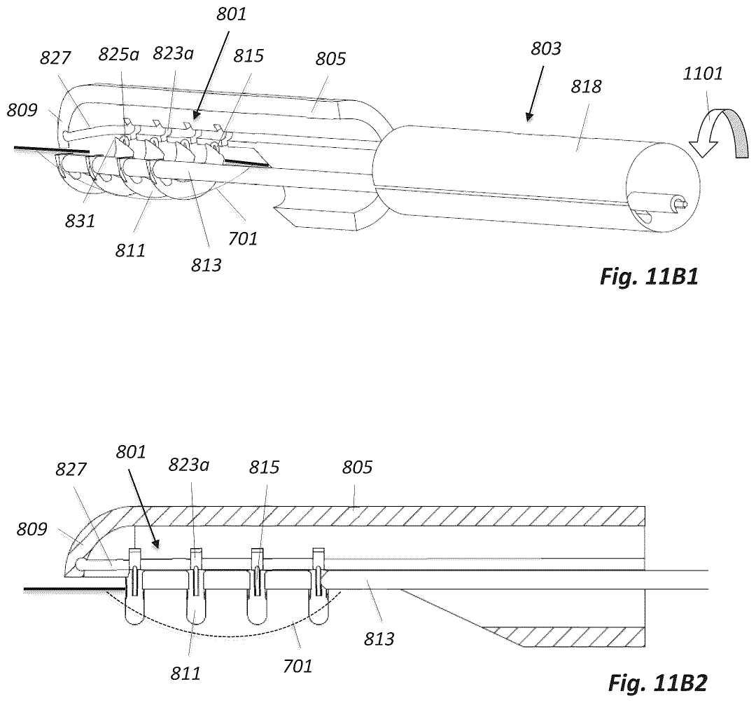

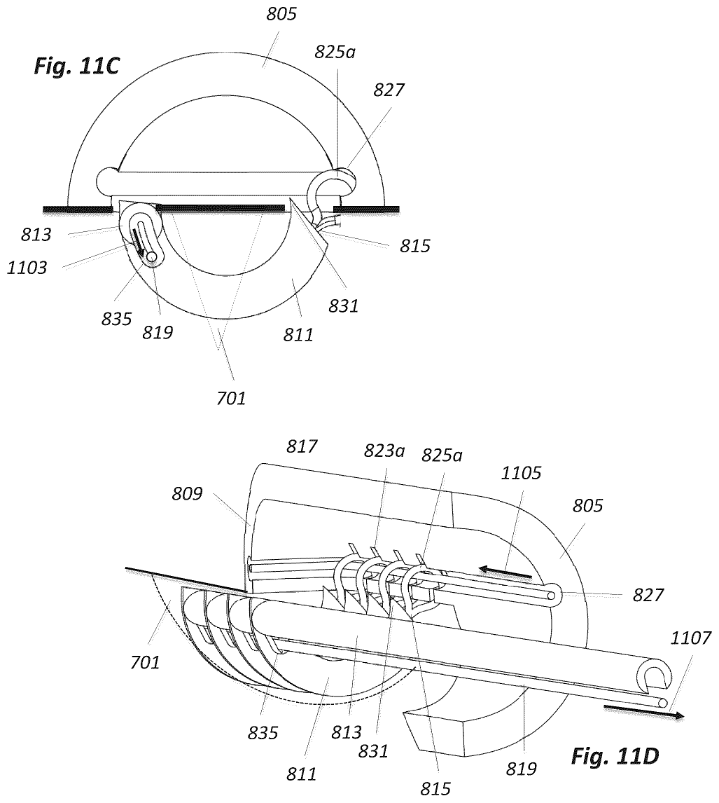

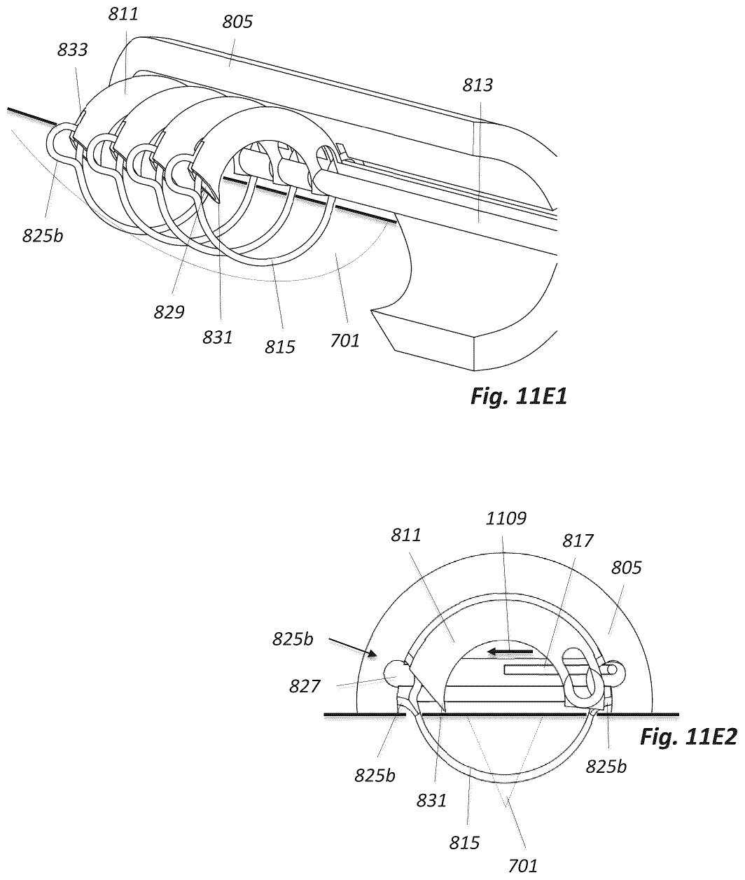

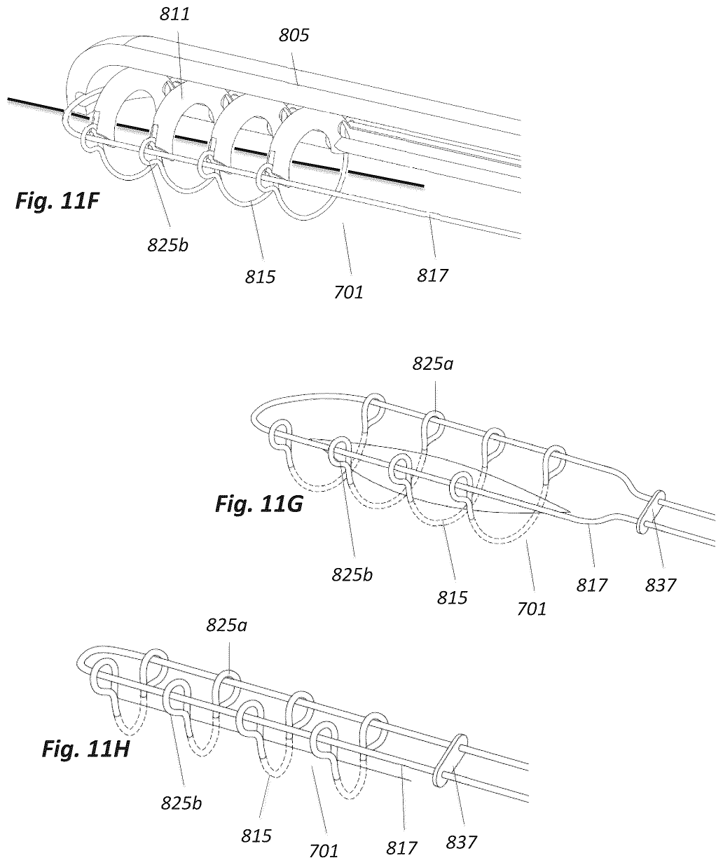

Provided in FIGS. 11A1 to 11H is an example method of utilizing a system for repairing tissue injury and/or closing an opening. A delivery catheter can deliver the repair system to the general area of the injury and/or opening. At that point, catheter 805 and suturing system 801 is advanced to the site of injury and/or opening, as shown in FIGS. 11A1 and 11A2.

In some implementations, elongated aperture 807 can then be positioned over injury and/or opening 701 such that catheter distal tip 809 is located near one end of the injury and/or opening and flex hinge 820 is located near the other end of the injury and/or opening. Suturing system 801 is delivered in an initial state. Accordingly, needles 811 are resting above injury and/or opening 701 with the needle tip 831 in contact with or near the tissue with traversing sutures 815 within the needle's inner lumen 829.

Upon turning torqueing rod 818 of suturing driver 803 in a counterclockwise direction 1101, suturing system 801 is actuated. FIGS. 11B1 and 11B2 show the result of turning torqueing rod 818 and actuating the suturing system. Because needles 811 are in connection with torqueing rod 818 via the driving needle holder 813, the needles also move in a counterclockwise direction to penetrate tissue surface 703 and traverse the injury and/or opening 701 and the associated tissue. Needle tip 831 traverses all the way through the injury and/or opening and penetrates the tissue surface on the other side of the injury and/or opening while the body of needles remain within the injury and/or opening and surrounding tissue. The movement of needles 811 carries the traversing sutures 815 within inner lumen 829 through the injury and/or opening and the tissue.

With needles 811 traversed and resting within injury and/or opening 701 and the surrounding tissue, suture loops 825a are actuated by suture positioning shaft 819 to open up and out of inner lumen 829 and slide into and fit within suture loop notches 823a (FIG. 11C). To actuate suture loops 825a, suture positioning shaft 819 is moved in the counterclockwise direction 1103 within suture positioning channel 835. Because suture positioning shaft 819 is within suture loops 825b, the movement of suture positioning shaft 819 pushes sutures 815 through inner lumen 829, pushing out suture loops 825a. Once out of the aperture, suture loops 825a open outward and fit within suture loop notches 823a such that the inner circumference of suture loops 825a are in line with final suture channel 827. With suture loops 825a open and in position, final suture 817 is advanced distally 1105 through final suture channel 827 and each of suture loops 825a to the other side of the loops, near or within distal tip 809 (FIG. 11D). Further, suture positioning shaft 819 is retrieved proximally 1107 and removed out of catheter 805.

With suture positioning shaft 819 removed and final suture 817 traversed through suture loops 825a, torqueing rod 818 can turn back in a clockwise direction to turnback needles 811 via the needle holder 813 such that the needles reversing back through the tissue and injury and/or opening until positioned above the injury and/or opening. As needles 811 reverse back, final suture 817 holds suture loops 825a and prevents traversing sutures 815 from reversing back and thus traversing sutures remain traversed through the injury and/or opening and surrounding tissue (FIGS. 11E1 and 11E2). Furthermore, as the needles 811 turn back, suture loop slit 833 grasps suture loops 825b such that the suture loops open outward and fit within suture loop notches 823b and the inner circumference of suture loops 825a are in line with final suture channel 827. With suture loops 825b open and in position, final suture 817 is advanced 1109 across and around final suture channel 827 within catheter distal tip 809 and then further advanced proximally on the other side of the catheter through each of suture loops 825b (FIG. 11F).

Upon final suture 817 traversing through suture loops 825b, catheter 805 can be removed off of the injury, opening, and/or tissue and returned proximally such that only traversing sutures 815 and the final suture remain (FIG. 11G). Accordingly, traversing sutures 815 traverse through the injury and/or opening and tissue with suture loops 825a and 825b just above the tissue surface on opposite sides of the injury and/or opening. Final suture rests within suture loops 825a and 825b and provides a means for cinching traversing sutures 815 such that they can come together and close the injury and/or opening. As shown in FIG. 11G, a pledget 837 is positioned onto final suture 817 and advanced distally to cinch traversing sutures 815. The cinching mechanism results in closing of injury and/or opening 701 (FIG. 11H). It should be understood that any mechanism for cinching the traversing sutures 815 via final suture 817 can be utilized. Final suture can be tied off or otherwise secured.

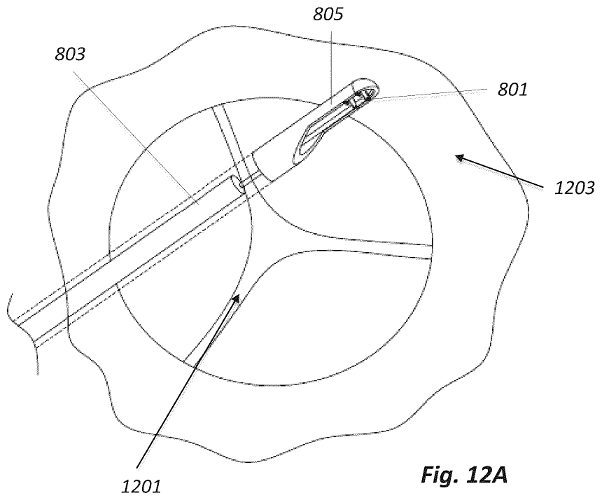

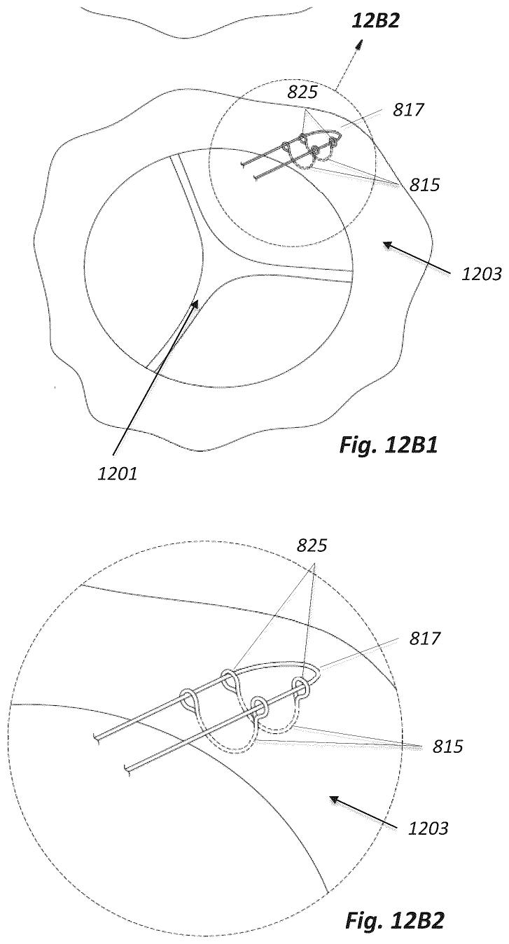

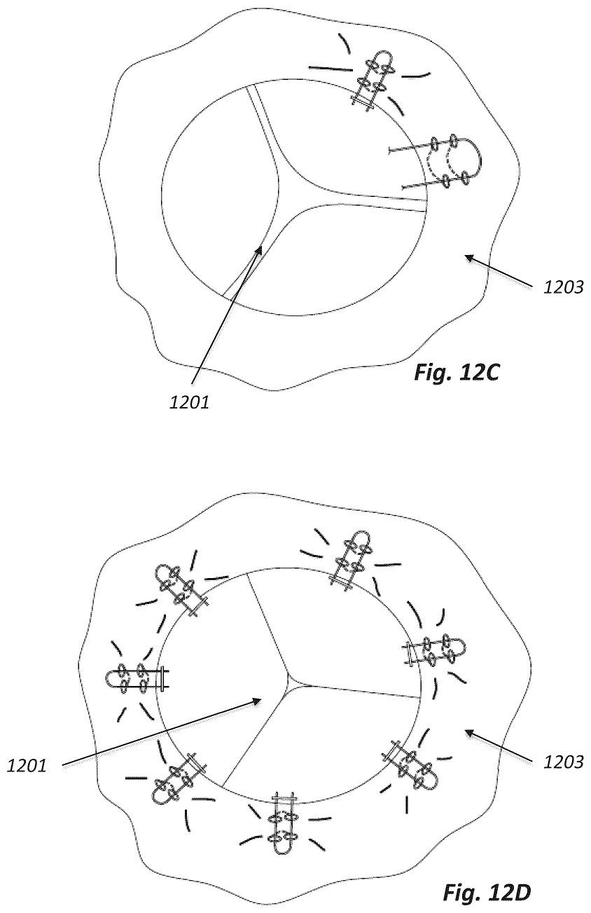

Provided in FIGS. 12A to 12D is an example method of utilizing a system for performing annuloplasty on a regurgitant valve. A delivery catheter can deliver the repair system to the general area of a regurgitant valve 1201. At that point, catheter 805 and suturing system 801 is advanced to the valve annulus 1203, as shown in FIG. 12A.

In some implementations, suturing system 801 is delivered in an initial states such that needles above annular tissue with the needle tip in contact with or near the tissue with traversing sutures within the needle's inner lumen. Suturing system 801 and suturing driver can operate as described in FIGS. 11A1 to 11F to yield.

Upon final suture 817 traversing through suture loops 825, catheter 805 can be removed off of the annular tissue and returned proximally such that only traversing sutures 815 and the final suture remain (FIGS. 12B1 and 12B2). Accordingly, traversing sutures 815 traverse through the annular tissue with suture loops 825 just above the tissue surface at an initial distance apart. Final suture rests within suture loops 825 and provides a means for cinching traversing sutures 815 such that they can come together and tighten the tissue. The cinching mechanism results in tightening of annular tissue 1203 (FIG. 12C). It should be understood that any mechanism for cinching the traversing sutures 815 via final suture 817 can be utilized. Final suture can be tied off or otherwise secured. Tightening of the tissue results in reducing the annular diameter, bringing the leaflets of the valve closer together to encourage better coaptation and reduction of regurgitation.

In some implementations, the catheter and suturing system is delivered a subsequent time to a second area of the valve annulus 1203 (FIG. 12C). The suturing and cinching of sutures are repeated at the second area to further tighten the annular tissue. The process of suturing and cinching of sutures can be repeated a number of times as necessary to tighten the annular tissue such that regurgitation is mitigated and/or eliminated (FIG. 12D).

Example 1. A system for treating prolapsed/bulging tissue, comprising:
a transcatheter system, wherein the transcatheter system comprises a vacuum catheter, a needle assembly, and a suture;
wherein the vacuum catheter comprises an inner lumen for providing a vacuum force and set of channels in a sidewall of the vacuum catheter;
wherein the set of channels allow distal advancement and proximal retrieval of the needle assembly.

Example 2. The system of example 1, wherein the needle assembly comprises a needle and a pin for suturing the suture.

Example 3. The system of example 1, wherein the vacuum catheter further comprises:
a set of ports, wherein each channel is connected with at least one port, and wherein each port faces the inner lumen.

Example 4. The system of example 3, wherein the set of channels comprises an initial channel and a final channel.

Example 5. The system of example 4, wherein the set of channels comprises one or more intermediate channels.

Example 6. The system of example 4, wherein each intermediate channel is in connection with an entry port and an exit port, and wherein the entry port and the exit port are connected via a connector channel.

Example 7. The system of example 6, wherein each connector channel comprises a slit-shaped aperture facing the inner lumen.

Example 8. The system of any one of examples 6-7, wherein each entry port of each intermediate channel is across an exit port on the opposite of the inner lumen.

Example 9. The system of any one of examples 6-8, wherein the initial channel comprises an exit port.

Example 10. The system of example 9, wherein the exit port of the initial channel is across an entry port of an intermediate channel.

Example 11. The system of any one of examples 6-10, wherein the final channel comprises an entry port.

Example 12. The system of example 11, wherein the entry port of the final channel is across an exit port of an intermediate channel.

Example 13. The system of example 3, wherein each channel has a companion channel across the inner lumen to yield a set of pairs of channels.

Example 14. The system of example 13, wherein each pair of channels of the set comprises a first channel that comprises an exit port and a second channel that comprises an entry port.

Example 15. The system of example 14, wherein the exit port of the first channel is across the inner lumen from the entry port of the second channel.

Example 16. The system of any one of examples 2-15, wherein the pin comprises a means for grasping the suture at a distal end of the pin.

Example 17. The system of any one of examples 2-16, wherein the pin is composed of a material with shape memory.

Example 18. The system of any one of examples 2-17, wherein the needle comprises an inner lumen capable of allowing the pin to traverse therethrough.

Example 19. The system of any one of examples 2-18, wherein the needle comprises a distal end that is sharpened, heated, or provides radiofrequency ablation.

Example 20. The system of any one of examples 1-19, wherein the transcatheter system is sterilized and stored within a container.

Example 21. A method for treating prolapsed/bulging tissue, comprising:
advancing a transcatheter system to tissue, wherein the transcatheter system comprises a vacuum catheter, a first needle assembly, and a suture, wherein the first needle assembly comprises a needle and a pin, wherein the vacuum catheter comprises an inner lumen for providing a vacuum force and set of channels in a sidewall of the vacuum catheter, wherein the set of channels allow distal advancement and proximal retrieval of the first needle assembly, and wherein each channel of the set comprises a port facing the inner lumen;
contacting the tissue with the vacuum catheter;
applying a vacuum force via the inner lumen of the vacuum catheter such that the tissue is constrained therein; and
suturing the tissue with the suture utilizing the needle assembly and the set of channels.

Example 22. The method of example 21, wherein the suturing step comprises distally advancing the first needle assembly through a channel of the set of channels and through its port to the tissue.

Example 23. The method of any one of examples 21-22, wherein the suturing step comprises traversing the tissue with the first needle assembly, wherein the first needle assembly carries the suture such that it traverses the tissue.

Example 24. The method of any one of examples 21-23, wherein the set of channels comprises an initial channel and a final channel; wherein the suturing step comprises:
distally advancing the first needle assembly through the initial channel;
traversing the first needle assembly through a port of the initial channel, and the tissue, wherein the first needle assembly carries the suture such that it traverses the port of the initial channel and the tissue;
grasping the suture with a final pin within a final channel; and proximally retracting the final pin, wherein the final pin carries the suture as it is proximally retracted.

Example 25. The method of example 24 further comprising:
securing a proximal end of the suture to the tissue; and securing a distal end of the suture to the tissue.

Example 26. The method of example 25, wherein the proximal end of the suture is preassembled to include a knot or a pledget.

Example 27. The method of any one of examples 25-26, wherein the final pin includes a means for tying off the distal end of the suture.

Example 28. The method of any one of examples 25-26, wherein the final pin includes a means for securing a pledget to the distal end of the suture.

Example 29. The method of any one of examples 24-28, wherein the set of channels comprises an intermediate channel; wherein the intermediate channel is in connection with an entry port and an exit port; wherein the entry port and the exit port are connected via a connector channel; wherein the suturing step comprises:
advancing the first needle assembly through the entry port of the intermediate channel, wherein the first needle assembly carries the suture such that it traverses the entry port of the intermediate channel;
distally advancing a second needle assembly through the intermediate channel, wherein the second needle assembly comprises a needle and a pin;
grasping the suture with the pin of the second needle assembly within the intermediate channel; and
traversing the second needle assembly through the exit port of the intermediate channel and the tissue, wherein the second needle assembly carries the suture such that it traverses the exit port of the intermediate channel and the tissue.

Example 30. The method of any one of examples 21-29, wherein the tissue is part of the cardiovascular system, wherein the method further comprises reconstituting the cardiovascular system with blood or saline.

Example 31. A system for transcatheter suturing, the system comprising:
a suturing catheter comprising an elongated aperture at a distal end of the suturing catheter and a closed distal tip; and
a suturing system situated within the elongated aperture, the suturing system comprising a plurality of needles, each needle having a needle tip and an inner lumen with a traversing suture within the inner lumen, wherein each of the needles are attached to a needle holder.

Example 32. The system of example 31, wherein the suturing catheter further comprises a flex hinge positioned at a proximal end of the elongated aperture.

Example 33. The system of example 31 further comprising:
a suturing driver situated proximally positioned relative to the suturing the system, wherein the suturing driver comprises a torqueing rod that is in connection with the needles via the needle holder, and wherein circular turning of the torqueing rod results in the needles turning in a circular motion.

Example 34. The system of example 33, wherein the suturing driver is within the suturing catheter.

Example 35. The system of example 33, wherein the suturing driver is extracorporeal.

Example 36. The system of any one of examples 31-35, wherein each traversing suture comprises a suture loop at the ends of the suture.

Example 37. The system of example 36, wherein the suturing catheter comprises a final suture channel and a plurality of notches within its side wall, wherein each notch is designed to fit and position the suture loop therein such that the suture loop is aligned with an axis of the final suture channel.

Example 38. The system of example 37 further comprising a final suture configured traverse the final suture channel and each suture loop when aligned with the axis of the final suture channel.

Example 39. The system of example 38 further comprising a pledget for cinching and securing the final suture.

Example 40. The system of example 37 further comprising a suture positioning shaft that is situated near and extended along the needle holder, wherein the suture positioning shaft is capable of positioning a suture loop within its respective notch via a circular motion of the suture positioning shaft.

Example 41. The system of example 40, wherein each needle comprises a suture positioning channel, wherein the positioning shaft traverses through each suture positioning channel, and wherein each suture positioning channel limits the circular motion of the suture positioning shaft.

Example 42. The system of example 40, wherein the suture positioning shaft traverses through the suture loops that are to be positioned within its respective notch.

Example 43. The system of any one of examples 31-42, wherein each needle of the plurality of needles comprises a tip that is sharpened, heated, or provides radiofrequency ablation.

Example 44. The system of any one of examples 31-43 further comprising an extracorporeal controller in connection with the suturing system.

Example 45. The system of any one of examples 31-44, wherein the suturing system is sterilized and stored within a container.

Example 46. A method for suturing tissue, the method comprising:
advancing a transcatheter system to a tissue site to be sutured, wherein the transcatheter system comprises a suturing catheter, a suturing system, and a suturing driver;
wherein the suturing catheter comprises an elongated aperture at a distal end of the suturing catheter, a closed distal tip, and a final suture channel;
wherein the suturing system is situated within the elongated aperture and comprises a plurality needles, each needle having a needle tip and an inner lumen with a traversing suture within the inner lumen, wherein each of the needles are attached to a needle holder, and wherein each traversing suture has first suture loop on one end of the suture and second suture loop on the other end of the suture;
wherein the suturing driver is situated proximally positioned relative to the suturing the system, wherein the suturing driver comprises a torqueing rod that is in connection with the needles via the needle holder; and
situating the elongated aperture and suturing system over the tissue site to be sutured.

Example 47. The method of example 46, further comprising turning the torqueing rod in a first circular motion such that the plurality of needles penetrates and traverses through the tissue site to be sutured, wherein the circular motion of the needles results in the each of the traversing sutures to traverse through the tissue site to be sutured.

Example 48. The method of example 47, wherein each first suture loop is positioned to align with the final suture channel.

Example 49. The method of any one of examples 46-48 further comprising passing a final suture through the final suturing channel and through each first suture loop.

Example 50. The method of any one of examples 47-49 further comprising turning the torqueing rod in a second circular motion that is opposite to the first circular motion such that the plurality of needles retreat back through the tissue site to be sutured; wherein the final suture that is traversed through each first suture loop keeps a plurality of traversing sutures traversed through the tissue site to be sutured; and wherein each second suture loop is positioned to align with the final suture channel.

Example 51. The method of any one of examples 46-50, further comprising passing the final suture through the final suturing channel and through each second suture loop.

Example 52. The method of any one of examples 46-51, further comprising cinching the plurality of traversing sutures via the final suture.

Example 53. The method of example 52, wherein the plurality of traversing sutures is cinched via a pledget.

Example 54. The method of any one of examples 46-53, wherein the suturing system further comprises a suture positioning shaft that is situated near and extended along the needle holder, wherein the suture positioning shaft traverses through the suture loops; and wherein the positioning of each first suture loop is positioned by moving the suture positioning shaft.

Example 55. The method of any one of examples 46-54, further comprising utilizing a transfemoral approach, a subclavian approach, a transapical approach, or a transaortic approach to reach the tissue site to be sutured.

Example 56. The method of any one of examples 46-55, wherein the tissue site to be sutured is an injury.

Example 57. The method of example 56, wherein the injury is a rupture, a tear, or an aneurism.

Example 58. The method of any one of examples 46-55, wherein the tissue site to be sutured is an incision.

Example 59. The method of any one of examples 46-55, wherein the tissue site to be sutured is annular tissue of a native valve.

Example 60. The method of example 59, wherein the native valve is regurgitant.

What is claimed is:

1. A system for transcatheter suturing, the system comprising:
a suturing catheter comprising an elongated aperture at a distal end of the suturing catheter and a closed distal tip; and
a suturing system situated within the elongated aperture, the suturing system comprising a plurality of needles, each needle having a needle tip and an inner lumen with a traversing suture within the inner lumen, wherein each of the needles are attached to a needle holder.

2. The system of claim 1, wherein the suturing catheter further comprises a flex hinge positioned at a proximal end of the elongated aperture.

3. The system of claim 1 further comprising:
a suturing driver situated proximally positioned relative to the suturing the system, wherein the suturing driver comprises a torqueing rod that is in connection with the needles via the needle holder, and wherein circular turning of the torqueing rod results in the needles turning in a circular motion.

4. The system of claim 3, wherein the suturing driver is within the suturing catheter.

5. The system of claim 3, wherein the suturing driver is extracorporeal.

6. The system of claim 1, wherein each traversing suture comprises a suture loop at the ends of the suture.

7. The system of claim 6, wherein the suturing catheter comprises a final suture channel and a plurality of notches within its side wall, wherein each notch is designed to fit and position the suture loop therein such that the suture loop is aligned with an axis of the final suture channel.

8. The system of claim 7 further comprising:
a final suture configured traverse the final suture channel and each suture loop when aligned with the axis of the final suture channel; and
a pledget for cinching and securing the final suture.

9. The system of claim 7 further comprising a suture positioning shaft that is situated near and extended along the needle holder, wherein the suture positioning shaft is capable of positioning a suture loop within its respective notch via a circular motion of the suture positioning shaft.

10. The system of claim 9, wherein each needle comprises a suture positioning channel, wherein the positioning shaft traverses through each suture positioning channel, and wherein each suture positioning channel limits the circular motion of the suture positioning shaft.

11. The system of claim 9, wherein the suture positioning shaft traverses through the suture loops that are to be positioned within its respective notch.

12. The system of claim 1 further comprising an extracorporeal controller in connection with the suturing system.

13. A method for suturing tissue, the method comprising:
advancing a transcatheter system to a tissue site to be sutured, wherein the transcatheter system comprises a suturing catheter, a suturing system, and a suturing driver;
wherein the suturing catheter comprises an elongated aperture at a distal end of the suturing catheter, a closed distal tip, and a fin
roximally positioned relative to the suturing the system, wherein the suturing driver comprises a torqueing rod that is in connection with the needles via the needle holder; and
situating the elongated aperture and suturing system over the tissue site to be sutured.

14. The method of claim 13, further comprising turning the torqueing rod in a first circular motion such that the plurality of needles penetrates and traverses through the tissue site to be sutured, wherein the circular motion of the needles results in the each of the traversing sutures to traverse through the tissue site to be sutured, wherein each first suture loop is positioned to align with the final suture channel.

15. The method of claim 13 further comprising passing a final suture through the final suturing channel and through each first suture loop.

16. The method of claim 14 further comprising turning the torqueing rod in a second circular motion that is opposite to the first circular motion such that the plurality of needles retreat back through the tissue site to be sutured; wherein the final suture that is traversed through each first suture loop keeps a plurality of traversing sutures traversed through the tissue site to be sutured; and wherein each second suture loop is positioned to align with the final suture channel.

17. The method of claim 13, further comprising passing the final suture through the final suturing channel and through each second suture loop.

18. The method of claim 13, further comprising cinching the plurality of traversing sutures via the final suture, wherein the plurality of traversing sutures is cinched via a pledget.

19. The method of claim 13, wherein the suturing system further comprises a suture positioning shaft that is situated near and extended along the needle holder, wherein the suture positioning shaft traverses through the suture loops; and wherein the positioning of each first suture loop is positioned by moving the suture positioning shaft.

20. The method of claim 13, further comprising utilizing a transfemoral approach, a subclavian approach, a transapical approach, or a transaortic approach to reach the tissue site to be sutured.
